(19) United States
(12) Patent Application Publication
(10) Pub. No.: US 2026/0256741 A1
(43) Pub. Date: Sep. 3, 2026

(54) ANTICANCER MALEIMIDE DERIVATIVES FOR USE WITH IMMUNE CHECKPOINT BLOCKADE

(71) Applicant: BROWN UNIVERSITY, Providence, RI (US)

(72) Inventors: Wafik El-Deiry, Providence, RI (US); Kelsey Huntington, Providence, RI (US)

(21) Appl. No.: 18/841,719

(22) PCT Filed: Mar. 8, 2023
(86) PCT No.: PCT/US2023/014817

§ 371 Date: Aug. 27, 2024

Related U.S. Application Data
(60) Provisional application No. 63/317,659, filed on Mar. 8, 2022.

(51) Int. Cl. A61K 31/407; A61K 39/00; A61K 39/395; A61P 35/00

(52) U.S. Cl. CPC A61K 31/407; A61K 39/3955; A61P 35/00; A61K 2039/505

(57) ABSTRACT

A method of treating cancer in a subject in need thereof is described. The method includes administering to the subject a therapeutically effective amount of a maleimide derivative while also treating the subject with an immune checkpoint blockade.

### CROSS-REFERENCE TO RELATED APPLICATIONS

This application claims priority to U.S. Provisional Application Ser. No. 63/317,659, filed on Mar. 8, 2022, which is hereby incorporated by reference in its entirety.

### GOVERNMENT FUNDING

The present invention was made with government support under Grant No. 1F31CA271636-01 awarded by the National Institutes of Health. The US government has certain rights in this invention.

### BACKGROUND

Glycogen synthase kinase 3 (GSK-3) is a serine/threonine kinase with key roles in myriad biological processes such as tumor progression, and inhibition of GSK-3 using a novel small-molecule elraglusib has shown promising preclinical antitumor activity in multiple tumor types. Ding et al., Clin Cancer Res., 25(21):6452-62 (2019). There is a growing body of literature characterizing the immunomodulatory roles of GSK-3 in the context of anti-tumor immunity. Augello et al., Cells, 9(6), 1427 (2020). GSK-3 is known to inhibit cytokine production and T cell activation. Tsai et al., Cells, 9(6), 1424 (2020). Aberrant overexpression of GSK-3 has been shown to promote tumor growth and epithelial-to-mesenchymal transition (EMT) through various mechanisms including modulation of pro-survival NF-κB signaling pathways. Mancinelli et al., Oxid Med Cell Longev., 2017:4629495 (2017). Thus, GSK-3 is a promising target in the treatment of human malignancies.

Globally, colorectal cancer (CRC) ranks third in incidence and second in mortality. Treatment options include surgery, chemotherapy, radiation therapy, targeted therapy, and immunotherapy. Immune checkpoint blockade (ICB) has now entered into clinical care for CRC with the recent U.S. Food and Drug Administration approvals of checkpoint inhibitors nivolumab and pembrolizumab for microsatellite instability-high (MSI-H) CRC cases after chemotherapy. Borelli et al., Cancers (Basel), 14(20), 4974 (2022). Thus far, ICB clinical trials have demonstrated efficacy in MSI-H CRC, however, the impressive durability of tumor regression stands in stark contrast with the lack of response observed in microsatellite stable (MSS) CRC. Thus, there remains a substantial unmet need in the ~85% of patients with MSS CRC in whom ICB is less effective. Gupta et al., Current Problems in Cancer, 42(6):548-59 (2018). Moreover, the percentage of patients with MSS CRC dramatically increases to ~96% in Stage IV disease.

### SUMMARY OF THE INVENTION

The ability to upregulate PD-L1 expression in tumor cells may be useful in promoting efficacy of anti-PD-1/L1 therapy. Inhibition of GSK-3 using small-molecule maleimide derivative elraglusib has shown promising preclinical antitumor activity. Using in vitro systems, the inventors found that elraglusib promotes immune cell-mediated tumor cell killing, enhances tumor cell pyroptosis, decreases tumor cell NF-κB-regulated survival protein expression, and increases immune cell effector molecule secretion. Using in vivo systems, the inventors observed synergy between elraglusib and anti-PD-L1 treatment in an immunocompetent murine model of colorectal cancer. Murine responders had more tumor-infiltrating T-cells, fewer tumor-infiltrating Tregs, lower tumorigenic circulating cytokine concentrations, and higher immunostimulatory circulating cytokine concentrations. To determine the clinical significance, human plasma samples were used from patients treated with elraglusib and correlated cytokine profiles with survival. Using paired tumor biopsies, it was found that CD45+ tumor-infiltrating immune cells had lower expression of inhibitory immune checkpoints and higher expression of T-cell activation markers in post-elraglusib patient biopsies. These results introduce several immunomodulatory mechanisms of GSK-3 inhibition using elraglusib, providing a rationale for the clinical evaluation of maleimide derivatives such as elraglusib in combination with immunotherapy for cancer treatment.

### BRIEF DESCRIPTION OF THE FIGURES

FIGS. 1A-10 provide graphs and images showing elraglusib triggers pyroptosis and sensitizes tumor cells to increase immune-mediated cytotoxicity in a co-culture model. Co-cultures were treated with drug concentrations as indicated. A 1:1 effector to target (E:T) ratio was used with a 24-hour co-culture duration. EthD-1 was used to visualize dead cells, 10× magnification, scale bar indicates 100 μm. (A) SW480 and TALL-104 T cell co-culture assay images at the 24-hour timepoint. 24-hour tumor cell pre-treatment with 5 μM elraglusib, followed by 24-hour co-culture. (B) Quantification of co-culture experiment using the percentage of dead cells out of total cells (n=3). (C) Quantification normalized by cell death observed with drug treatment alone (n=3). (D) SW480 and donor-derived CD8+ T cell co-culture assay images at the 24-hour timepoint. 24-hour tumor cell pre-treatment with 5 μM elraglusib, followed by 24-hour co-culture. (E) Quantification of co-culture experiment using the percentage of dead cells out of total cells (n=3). (F) Quantification normalized by cell death observed with drug treatment alone (n=3). (G) The number of HCT 116 GFP+ cells were quantified after 48 hours of culture with DMSO, 5 μM elraglusib, and/or 5000 TALL-104 cells (n=3). (H) The number of HT-29 GFP+ cells were quantified after 48 hours of culture with DMSO, 5 μM elraglusib, and/or 5000 TALL-104 cells (n=3). (I) The number of HCT 116 GFP+ cells were quantified after 48 hours of culture with DMSO, 5 μM elraglusib, and/or 5000 NK-92 cells (n=3). (J) The number of HT-29 GFP+ cells were quantified after 48 hours of culture with DMSO, 5 μM elraglusib, and/or 5000 NK-92 cells (n=3). (K) 40× images were collected with a Molecular Devices ImageXpress® Confocal HT.ai High-Content Imaging System. White arrows indicate pyroptotic events. Western blot analysis of (L) HCT-116 and (M) HT-29 CRC cells for expression of indicated proteins after treatment with indicated cytokines or drugs. Quantification of IFN-γ secretion (pg/mL) post-DMSO or elraglusib treatment for 24 hours in (N) TALL-104 cells and (O) NK-92 cells (n=3). Error bars represent the mean+/−standard deviation. Statistical test: one-way ANOVA with Tukey's test for multiple comparisons. P-value legend: * p<0.05, ** p<0.01, *** p<0.001, **** p<0.0001.

FIGS. 2A-21 provide graphs and images showing elraglusib treatment induces apoptosis and suppresses survival pathways in tumor cells. Western blot analysis of (A) HCT-116 and HT-29 CRC cells for expression of indicated proteins after increasing durations of elraglusib treatment (0-72 hours). (B) Western blot analysis of Mcl-1 expression in HCT-116 CRC cells after increasing durations of elraglusib treatment. CRC cells were treated with 1 μM elraglusib for 24 hours and treated versus untreated samples were compared in triplicate. Microarray analysis results were visualized using volcano plots for (C) HCT-116, (D) HT-29, and (E) KM12C CRC cell lines. Top down- and up-regulated genes post-elraglusib treatment as compared to controls are shown. Results were calculated using a FC cutoff of >1.5, <−1.5, and ap value of <0.05. (F) The number of genes up- or down-regulated in each of the three cell lines within several signaling pathways of interest. Green indicates downregulation and red indicates upregulation of gene expression. (G) A Venn Diagram was used to compare the 3,124 genes that were differentially expressed post-treatment with elraglusib in the three colon cancer cell lines (HCT-116, HT-29, KM12C). (H) Tumor cells (HCT-116, HT-29) were treated with 1 μM elraglusib for 48 hours and cell culture supernatant was analyzed with the Luminex 200. Red indicates a positive FC and green indicates a negative FC (n=3). (I) Tumor cells (HCT-116, HT-29) were treated with 5 μM elraglusib for 48 hours and cell culture supernatant was analyzed with the Luminex 200. Red indicates a positive FC and green indicates a negative FC (n=3).

FIGS. 3A-3J provide graphs and images showing elraglusib treatment increases effector molecule secretion and induces an energetic shift in cytotoxic immune cells. Western blot analysis of (A) TALL-104 and (B) HT-29 cytotoxic immune cells for expression of indicated proteins after increasing durations of elraglusib treatment (0-72 hours). (C) Proposed model for non-canonical NF-κB pathway activation: increased NIK expression indicates non-canonical NF-κB pathway activation which enhances the expression of chemokines and cytokines (CCL11, TNF-α, GM-CSF) and subsequently leads to increased recruitment and proliferation of cytotoxic immune cells (CD8+ T, CD4+ T, NK cells). Immune cells were treated with 1 μM elraglusib for 24 hours and treated versus untreated samples were compared in triplicate. Microarray analysis results were visualized using volcano plots for (D) NK-92 and (E) TALL-104 immune cell lines. (F) A Venn Diagram was used to compare the 124 genes that were differentially expressed post-treatment with elraglusib in the two immune cell lines (TALL-104, NK-92). (G) 10× single-cell sequencing analysis of immune cells treated with elraglusib. TALL-104 and NK-92 cells were treated with 1 μM elraglusib for 24 hours and aggregate data was visualized using a t-SNE plot. (H) Immune cells show differential expression of mitochondria-encoded genes (MT) and ribosomal genes (RB) post-treatment with elraglusib. (I) Heatmap comparing gene expression post-elraglusib treatment as compared to control. Red indicates a positive FC and green indicates a negative FC. (J) Immune cells (TALL-104, NK-92) were treated with 1 μM elraglusib for 48 hours and cell culture supernatant was analyzed with the Luminex 200. Red indicates a positive FC and green indicates a negative FC (n=3).

FIGS. 4A-4M provide graphs and images showing elraglusib enhances immune cell tumor-infiltration to prolong survival in combination with anti-PD-L1 therapy in a syngeneic murine model of MSS colon carcinoma. (A) Experimental model overview of the syngeneic murine colon carcinoma BALB/c murine model using MSS cell line CT-26. (B) Kaplan-Meier estimator curves for all treatment groups as indicated. Statistical significance was determined using a Log-rank (Mantel-Cox) test. (C) Overview of cell lineage markers used for flow cytometric immunophenotyping analysis. 14-days post-treatment initiation immune cell subpopulations were analyzed in the spleen and tumor. (D) Splenic T cells, (E) Tumor-infiltrating T cells, (F) Splenic NK cells, and (G) Tumor-infiltrating NK cells were compared between responders (R, n=3) and non-responders (NR, n=18). NK cell subsets based on the expression of CD11b and CD27 were compared in the spleen and visualized via (H) bar graph and (I) pie chart. NK cell subsets based on the expression of CD11b and CD27 were also compared in the tumor and visualized via (J) bar graph and (K) pie chart. T cell ratios were compared in the (L) Spleen and (M) Tumor. Statistical significance was determined using two-tailed unpaired T tests. P-value legend: * p<0.05, ** p<0.01, *** p<0.001, **** p<0.0001.

FIGS. 5A-5W provide graphs and images showing responders have a more immunostimulatory tumor microenvironment as compared to non-responders. IHC analysis of tumors 14 days post-treatment initiation or tumors from long-term mice. Non-responders (NR) and responders (R) were compared. 20× images, scale bar represents 100 μm. (A-B) CD3, (C-D) Granzyme B, (E-F) Ki67, (G-H) PD-L1, and (I-J) cleaved-caspase 3 (CC3) were compared at the 14 days post-treatment initiation timepoint, and the long-term timepoint, respectively. Statistical significance was determined using two-tailed unpaired T tests (n=6). Serum from long-term mice sacrificed was analyzed via cytokine profiling for (K) CCL21, (L) VEGFR2, (M) CCL7, (N) CCL12, (O) BAFF, (P) VEGF, (Q) IL-1β, (R) IL-6, (S) CCL22, (T) GM-CSF, (U) CCL4, (V) TWEAK, and (W) CCL2. Responders (red) and non-responders (black) were compared. A Kruskal-Wallis test was used to calculate statistical significance followed by a Benjamini-Hochberg correction for multiple comparisons. p values are shown for analytes that were significantly different between responders and non-responders and are ordered by significance. P-value legend: * p<0.05, ** p<0.01, *** p<0.001, **** p<0.0001.

FIGS. 6A-6D provide graphs showing patient plasma concentrations of cytokines correlate with progression-free survival (PFS), overall survival (OS), and in vivo response to therapy results. Plasma samples from human patients with refractory solid tumors of multiple tissue origins enrolled in a Phase 1 clinical trial investigating a novel GSK-3 inhibitor elraglusib (NCT03678883) were analyzed using a Luminex 200 (n=19). (A) Table summarizing patient demographics. (B) Baseline and 24-hour post-elraglusib plasma concentrations of cytokines, chemokines, and growth factors were plotted against PFS and OS. Simple linear regressions were used to calculate significance. p values less than 0.05 were reported as statistically significant. (C) Cytokines grouped by function. Fold change (FC) is shown where green indicates a negative (<0) FC compared to the baseline (pre-dose) value and red indicates a positive (>0) FC. (D) Table comparing murine and human circulating biomarker trends. Red boxes indicate that an analyte concentration positively correlated with response to therapy/PFS/OS while green boxes indicate that an analyte concentration negatively correlated with response to therapy/PFS/OS.

FIGS. 7A-7H provide graphs and images showing spatial profiling of patient tumor biopsies reveals a more immunostimulatory tumor microenvironment post-treatment with elraglusib. Patient samples were analyzed using NanoString GeoMx Digital Spatial Profiling (DSP) technology. (A) Pie charts showing biopsy timepoint, primary tumor type, metastatic biopsy tissue type, and paired/unpaired biopsy sample information breakdowns. (B) A representative region of interest (ROI) showing PanCK+ and CD45+ masking. Green indicates CK, red indicates CD45, and blue indicates DAPI staining. (C) A Sankey diagram was used to visualize the study design where the width of a cord in the figure represents how many segments are in common between the two annotations they connect. The scale bar represents 50 segments. Blue cords represent CD45+ segments and yellow cords represent panCK+ segments. (D) Heatmap of all areas of interest (AOIs). Patient IDs, immune cell locations, biopsy timepoint, biopsy tissue, primary tumor location, and segment identity information are color coded as indicated in the legend. (E) PanCK+ ROI CD39 expression plotted against time-on-study (TOS). Points are color-coded by time on study (TOS)/time on treatment with darker blue points indicating a shorter TOS or time on treatment. (F) CD45+ ROI CD163 expression plotted against TOS. (G) Volcano plot showing a comparison of CD45+ region protein expression in post-treatment biopsies and pre-treatment biopsies regardless of timepoint. Grey points are non-significant (NS), blue points have p values <0.05, and red points have false discovery rate (FDR) values less than 0.05. The size of the point represents the log 2 UQ Signal-to-noise ratio (SNR). (H) Volcano plot showing a comparison of tumor-infiltrating CD45+ immune cell segment protein expression in pre-versus post-treatment biopsies. Grey points are non-significant (NS), blue points have p values <0.05, and red points have false discovery rate (FDR) values less than 0.05. The size of the point represents the log 2 UQ Signal-to-noise ratio (SNR).

### DETAILED DESCRIPTION OF THE INVENTION

The present invention provides a method of treating cancer in a subject in need thereof, comprising administering to the subject a therapeutically effective amount of a maleimide derivative while also treating the subject with an immune checkpoint blockade.

### Definitions

The terminology as set forth herein is for description of the embodiments only and should not be construed as limiting of the invention as a whole. As used in the description of the invention and the appended claims, the singular forms “a”, “an”, and “the” are inclusive of their plural forms, unless contraindicated by the context surrounding such.

As used herein, the term “organic group” is used to mean a hydrocarbon group that is classified as an aliphatic group, cyclic group, or combination of aliphatic and cyclic groups (e.g., alkaryl and aralkyl groups). In the context of the present invention, suitable organic groups for the compounds of this invention are those that do not interfere with the anti-cancer activity of the compounds. In the context of the present invention, the term “aliphatic group” means a saturated or unsaturated linear or branched hydrocarbon group. This term is used to encompass alkyl, alkenyl, and alkynyl groups, for example.

As used herein, the terms “alkyl”, “alkenyl”, and the prefix “alk-” are inclusive of straight chain groups and branched chain groups. Unless otherwise specified, these groups contain from 1 to 20 carbon atoms, with alkenyl groups containing from 2 to 20 carbon atoms. In some embodiments, these groups have a total of at most 10 carbon atoms, at most 8 carbon atoms, at most 6 carbon atoms, or at most 4 carbon atoms. Alkyl groups including 4 or fewer carbon atoms can also be referred to as lower alkyl groups. Alkyl groups can also be referred to by the number of carbon atoms that they include (i.e., C₁-C₄ alkyl groups are alky groups including 1-4 carbon atoms).

Cycloalkyl, as used herein, refers to an alkyl group (i.e., an alkyl, alkenyl, or alkynyl group) that forms a ring structure. Cyclic groups can be monocyclic or polycyclic and preferably have from 3 to 10 ring carbon atoms. A cycloalkyl group can be attached to the main structure via an alkyl group including 4 or less carbon atoms. Exemplary cyclic groups include cyclopropyl, cyclopropylmethyl, cyclopentyl, cyclohexyl, adamantyl, and substituted and unsubstituted bornyl, norbornyl, and norbornenyl.

Unless otherwise specified, “alkylene” and “alkenylene” are the divalent forms of the “alkyl” and “alkenyl” groups defined above. The terms, “alkylenyl” and “alkenylenyl” are used when “alkylene” and “alkenylene”, respectively, are substituted. For example, an arylalkylenyl group comprises an alkylene moiety to which an aryl group is attached.

The term “haloalkyl” is inclusive of groups that are substituted by one or more halogen atoms, including perfluorinated groups. This is also true of other groups that include the prefix “halo-”. Examples of suitable haloalkyl groups are chloromethyl, trifluoromethyl, and the like. Halo moieties include chlorine, bromine, fluorine, and iodine.

The term “aryl” as used herein includes carbocyclic aromatic rings or ring systems. Examples of aryl groups include phenyl, naphthyl, biphenyl, fluorenyl and indenyl. Aryl groups may be substituted or unsubstituted.

Unless otherwise indicated, the term “heteroatom” refers to the atoms O, S, or N. The term “heteroaryl” includes aromatic rings or ring systems that contain at least one ring heteroatom (e.g., O, S, N). In some embodiments, the term “heteroaryl” includes a ring or ring system that contains 3 to 12 carbon atoms, 1 to 3 rings, 1 to 4 heteroatoms, and O, S, and/or N as the heteroatoms. Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

The terms “arylene” and “heteroarylene” are the divalent forms of the “aryl” and “heteroaryl” groups defined above. The terms “arylenyl” and “heteroarylenyl” are used when “arylene” and “heteroarylene”, respectively, are substituted. For example, an alkylarylenyl group comprises an arylene moiety to which an alkyl group is attached.

When a group is present more than once in any formula or scheme described herein, each group (or substituent) is independently selected, whether explicitly stated or not. For example, for the formula —C(O)—NR₂ each R group is independently selected.

As a means of simplifying the discussion and the recitation of certain terminology used throughout this application, the terms “group” and “moiety” are used to differentiate between chemical species that allow for substitution or that may be substituted and those that do not so allow for substitution or may not be so substituted. Thus, when the term “group” is used to describe a chemical substituent, the described chemical material includes the unsubstituted group and that group with nonperoxidic O, N, S, Si, or F atoms, for example, in the chain as well as carbonyl groups or other conventional substituents. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, tert-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, cyanoalkyls, etc. On the other hand, the phrase “alkyl moiety” is limited to the inclusion of only pure open chain saturated hydrocarbon alkyl substituents, such as methyl, ethyl, propyl, tert-butyl, and the like.

The invention is inclusive of the compounds described herein in any of their pharmaceutically acceptable forms, including isomers (e.g., diastereomers and enantiomers), tautomers, salts, solvates, polymorphs, prodrugs, and the like. In particular, if a compound is optically active, the invention specifically includes each of the compound's enantiomers as well as racemic mixtures of the enantiomers. It should be understood that the term “compound” includes any or all of such forms, whether explicitly stated or not (although at times, “salts” are explicitly stated).

A subject, as defined herein, is an animal such as a vertebrate or invertebrate organism. In other embodiments, the subject is a mammal such as a domesticated farm animal (e.g., cow, horse, pig) or pet (e.g., dog, cat). More preferably, the subject is a human. A subject at risk is a subject who has been determined to have an above-average risk that a subject will develop cancer, which can be determined, for example, through family history or the detection of genes causing a predisposition to developing cancer.

Treat”, “treating”, and “treatment”, etc., as used herein, refer to any action providing a benefit to a subject at risk for or afflicted with a condition or disease such as cancer, including improvement in the condition through lessening or suppression of at least one symptom, delay in progression of the disease, prevention or delay in the onset of the disease, etc. The subject may be at risk due to exposure to carcinogenic agents, being genetically predisposed to disorders characterized by unwanted, rapid cell proliferation, and so on.

“Pharmaceutically acceptable” as used herein means that the compound or composition is suitable for administration to a subject for the methods described herein, without unduly deleterious side effects in light of the severity of the disease and necessity of the treatment.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses.

### Methods of Treating Cancer

The present invention provides a method of treating cancer in a subject in need thereof, comprising administering to the subject a therapeutically effective amount of a maleimide derivative while also treating the subject with an immune checkpoint blockade.

Novel maleimide-derivatives can be clinically used to improve patient response to immune checkpoint blockade. The inventors have demonstrated that combination therapy with these derivatives and immunotherapy will improve treatment response in patients who traditionally would respond poorly to immunotherapy, for example, patients having low PD-L1 expression in their tumors. The inventors have identified several maleimide-derivatives that have the potential to upregulate tumor cell PD-L1 expression for therapeutic synergy with immune checkpoint blockade.

The method comprises administering a maleimide derivative to a subject. The structures of maleimide derivatives that can be used in the method are shown by Formula I, below:

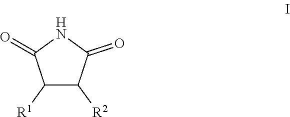

wherein R¹ and R² comprise an alkyl or aryl group. In some embodiments, the alkyl or aryl groups are C₆-C₁₂ alkyl or aryl groups. The alkyl or aryl groups can be substituted or unsubstituted. In some embodiments, R¹ and R² are C₆-C₁₂ aryl groups. In some embodiments, R¹ and R² are heteroaryl groups.

A maleimide derivative is a compound including the maleimide backbone shown in formula I, or a compound that can be readily imagined to arise from a known maleimide derivative if a group is attached to the compound or an atom or group is replace with another atom or group. Maleimide derivatives also include structural analogs of maleimide derivatives in which a small number of atoms in the structural backbone have been replaced with a similar atom, such as replacement of an oxygen or carbon atom with a nitrogen atom. A number of specific maleimide derivatives are described herein.

In some embodiments, R¹ and R² comprise phenyl and/or indolyl groups, providing an arylindolemaleimide derivative. Examples of arylindolemaleimide derivatives include SB-216763 and SB-41528, the structures of which are shown below:

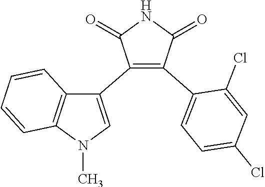 [SMILES: Cn1cc(C2=C(c3ccc(Cl)cc3Cl)C(=O)NC2=O)c2ccccc21]

SB-216763; 3-(2,4-dichlorophenyl)-4-(1-methyl-1H-indol-3-yl)-1H-pyrrole-2,5-dione.

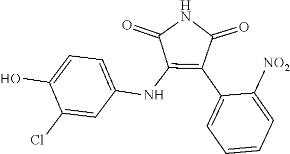 [SMILES: O=C1NC(=O)C(c2ccccc2[N+](=O)[O-])=C1Nc1ccc(O)c(Cl)c1]

SB-415286; 3-[(3-chloro-4-hydroxyphenyl)amino]-4-(2-nitrophenyl)-1H-pyrrole-2,5-dione.

In some embodiments, R¹ and R² comprise indoyl groups, thereby providing a bisindoylmaleimide derivative. In some embodiments, R¹ and R² comprise indoyl or benzofuranyl groups. In some embodiments, the maleimide derivative is a compound selected from the group consisting of NSC767225, NSC767202, NSC767238, NSC59984, NSC767276, NSC767334, NSC767335, 9-ING-41, 5-ING-135, 2-ING-173, 9-ING-49, and 9-ING-87. The chemical names and/or chemical structures of these maleimide derivatives are provided below:

NSC767225, 3-(1,2-dimethylindol-3-yl)-4-(1-methylindol-3-yl)pyrrole-2,5-dione; NSC767202, 3-(1-methylindol-3-yl)-4-(1-methyl-2-methylsulfinylindol-3-yl)pyrrole-2,5-dione; NSC767238, 3-(1-methylindol-3-yl)-4-(6,7,8,9-tetrahydropyrido[1,2-a]indol-10-yl)pyrrole-2,5-dione; NSC59984, (E)-1-(4-methylpiperazin-1-yl)-3-(5-nitrofuran-2-yl)prop-2-en-1-one; NSC767276, 3-[(11R,15R)-13-methyl-1,13-diazatetracyclo[7.7.0.02,7.011,15]hexadeca-2,4,6,8-tetraen-8-yl]-4-(1-methylindol-3-yl)pyrrole-2,5-dione; NSC767334, 3-(1,2-dimethylindol-3-yl)-4-(3-methoxyphenyl)pyrrole-2,5-dione; NSC767335, 3-(5-chloro-1-methylindol-3-yl)-4-(3-methoxyphenyl)pyrrole-2,5-dione.

9-ING-41,

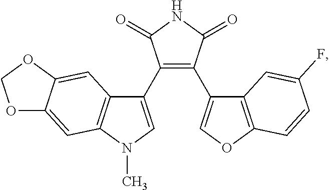 [SMILES: Cn1cc(C2=C(c3coc4ccc(F)cc34)C(=O)NC2=O)c2cc3c(cc21)OCO3]

3-(5-fluorobenzofuran-3-yl)-4-(5-methyl-5H-[1,3]dioxolo[4,5-f]indol-7-yl)-1H-pyrrole-2,5-dione.

5-ING-135,

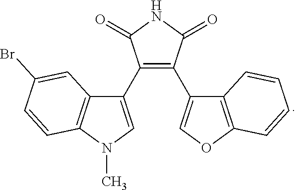 [SMILES: Cn1cc(C2=C(c3coc4ccccc34)C(=O)NC2=O)c2cc(Br)ccc21]

### 2-ING-173, CH₃

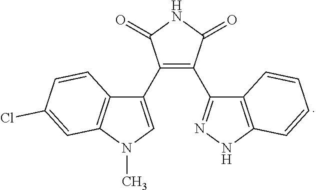 [SMILES: Cn1cc(C2=C(c3n[nH]c4ccccc34)C(=O)NC2=O)c2ccc(Cl)cc21]

9-ING-49,

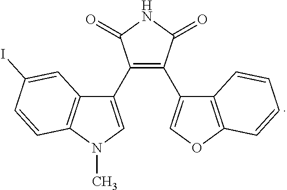 [SMILES: Cn1cc(C2=C(c3coc4ccccc34)C(=O)NC2=O)c2cc(I)ccc21]

And 9-ING-87

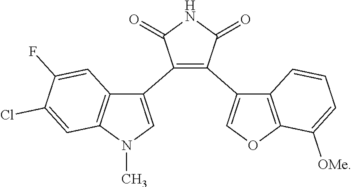 [SMILES: COc1cccc2c(C3=C(c4cn(C)c5cc(Cl)c(F)cc45)C(=O)NC3=O)coc12]

In some embodiments, the maleimide derivative is a glycogen synthase kinase-3 (GSK-3) inhibitor. In mammals, including humans, GSK-3 exists in two isozymes encoded by two homologous genes GSK-3a (GSK3A; Ref. Seq. NM_019884) and GSK-3B (GSK3B; Ref. Seq. NM_002093). A number of maleimide derivative GSK-3 inhibitors are known to those skilled in the art. See Gunosewoyo et al., J Med Chem., 56(12): 5115-5129 (2013); Ye et al., Eur J Med Chem., 68:361-71 (2013); Hilliard et al., Anticancer Drugs, 22(10): 978-985 (2011), and Gunosewoyo et al., J Med Chem., 56(12): 5115-5129 2013), the disclosures of which are incorporated herein by reference.

Cancer is generally named based on its tissue of origin. There are several main types of cancer. Carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma and multiple myeloma are cancers that begin in the cells of the immune system. Cancer which has metastasized will still retain traits associated with its tissue of origin. Examples of types of cancer that can be treated using the compounds of the present invention include cancer is selected from the group consisting of leukemia, hepatic cancer, non-small cell lung cancer, colon cancer, central nervous system cancer, melanoma, ovarian cancer, renal cancer, prostate cancer, and breast cancer.

In some embodiments, the cancer being treated is a low programmed death ligand 1 (PD-L1) expressing cancer. PD-1 is a checkpoint protein found on B cells, T cells, and myeloid cells. PD-L1 is a 40 kDa type 1 transmembrane protein on macrophages and some cancer cells that that reduces the proliferation of antigen-specific T-cells in lymph nodes while reducing apoptosis in regulatory T cells. upregulation of PD-L1 may allow cancers to evade the host immune system. For example, an analysis of 196 tumor specimens from patients with renal cell carcinoma found that high tumor expression of PD-L1 was associated with increased tumor aggressiveness and a 4.5-fold increased risk of death. Thompson et al., PNAS 101 (49): 17174-17179 (2004). PD-L1 levels may be detected using routine methods of detecting proteins known to those skilled in the art.

In some embodiments, the maleimide derivatives of the present invention are used to treat colorectal cancer. Colorectal cancer, also known as bowel cancer, colon cancer, or rectal cancer, is the development of cancer from the colon or rectum. Symptoms of colorectal cancer include worsening constipation, blood in the stool, decrease in stool caliber (thickness), loss of appetite, loss of weight, and nausea or vomiting in someone over 50 years old. However, almost 50% of individuals having colorectal cancer do not report any symptoms. Individuals with inflammatory bowel disease (ulcerative colitis and Crohn's disease) have an increased risk of developing colon cancer. Colorectal cancer can be imaged using a CT scan, PET imaging, or MRI, and is usually detected by analysis of a biopsy such as that obtained during a colonoscopy.

In some embodiments, the cancer being treated is microsatellite stable (e.g., microsatellite stable colorectal cancer). Microsatellite stable (MSS) describes the situation where the number of microsatellite repeats in DNA are the same in all cells of the body. MSS generally indicates a poor prognosis for a cancer patient, as these types of cancer are more difficult to treat.

The effectiveness of cancer treatment may be measured by evaluating a reduction in tumor load. The reduction in tumor load may be represent a direct decrease in mass, or it may be measured in terms of tumor growth delay, which is calculated by subtracting the average time for control tumors to grow over to a certain volume from the time required for treated tumors to grow to the same volume.

Candidate agents may be tested in animal models. Typically, the animal model is one for the study of cancer. The study of various cancers in animal models (for instance, mice) is a commonly accepted practice for the study of human cancers. For instance, the nude mouse model, where human tumor cells are injected into the animal, is commonly accepted as a general model useful for the study of a wide variety of cancers (see, for instance, Polin et al., Investig. New Drugs, 15:99-108 (1997)). Results are typically compared between control animals treated with candidate agents and the control littermates that did not receive treatment. Transgenic animal models are also available and are commonly accepted as models for human disease (see, for instance, Greenberg et al., Proc. Natl. Acad. Sci. USA, 92:3439-3443 (1995)). Candidate agents can be used in these animal models to determine if a candidate agent decreases one or more of the symptoms associated with the cancer, including, for instance, cancer metastasis, cancer cell motility, cancer cell invasiveness, or combinations thereof.

The inventors have determined that the maleimide derivatives can exhibit synergistic effects when used together with immune checkpoint blockade. Immune checkpoints are a normal part of the immune system, and prevent an immune response from being so strong that it destroys healthy cells in the body. For example, the binding of PD-L1 to PD-1 keeps T cells from killing tumor cells in the body. Another example of an immune checkpoint protein is CTLA-4. When the checkpoint and partner proteins bind together, they send an “off” signal to the T cells. Immunotherapy drugs called immune checkpoint inhibitors work by blocking checkpoint proteins from binding with their partner proteins.

The invention includes administration of a maleimide derivative while also treating the subject with an immune checkpoint blockade. Immune checkpoint blockade can be achieved in a variety of ways, including co-administration of an immune checkpoint inhibitor. Co-administration of an immune checkpoint inhibitor includes simultaneous administration, but also includes administration at a time proximal to administration (e.g., administration before or after) of a maleimide derivative, so long as the administration is close enough in time for the agents to have overlapping effects.

A wide variety of immune checkpoint inhibitors are known to those skilled in the art. See Postow et al., J Clin Oncol., 33(17): 1974-1982 (2015). Examples of immune checkpoint inhibitors include PD-1 inhibitors such as pembrolizumab, nivolumab, and cemiplamab, CTLA-4 inhibitors such as ipilimumab and tremelimumab, and LAG-3 inhibitors such as relatlimab. In some embodiments, the immune checkpoint blockade comprises PD-L1 inhibition. Examples of PD-L1 inhibitors include atezolizumab, avelumab, and durvalumab.

### Formulation and Administration of Anticancer Compounds

The present invention provides a method for administering one or more anti-cancer maleimide derivatives in a pharmaceutical composition. Accordingly, in some embodiments, the maleimide derivative is administered together with a pharmaceutically acceptable carrier. Examples of pharmaceutical compositions include those for oral, intravenous, intramuscular, subcutaneous, or intraperitoneal administration, or any other route known to those skilled in the art, and generally involves providing an anti-cancer compound formulated together with a pharmaceutically acceptable carrier.

When preparing maleimide derivatives or immune checkpoint inhibitors for oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable carrier.

For intravenous, intramuscular, subcutaneous, or intraperitoneal administration, the compound may be combined with a sterile aqueous solution which is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering said solution sterile. The formulations may be present in unit or multi-dose containers such as sealed ampoules or vials.

Formulations suitable for parenteral administration conveniently comprise a sterile aqueous preparation of the active compound which is preferably made isotonic. Preparations for injections may also be formulated by suspending or emulsifying the active compounds in non-aqueous solvent, such as vegetable oil, synthetic aliphatic acid glycerides, esters of higher aliphatic acids or propylene glycol.

The dosage form and amount can be readily established by reference to known treatment or prophylactic regiments. The amount of therapeutically active compound that is administered and the dosage regimen for treating a disease condition with the compounds and/or compositions of this invention depends on a variety of factors, including the age, weight, sex, and medical condition of the subject, the severity of the disease, the route and frequency of administration, and the particular compound employed, the location of the unwanted proliferating cells, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight, may be appropriate. The daily dose can be administered in one to four doses per day.

For example, the maximum tolerated dose (MTD) for anti-cancer compounds such as maleimide derivatives can be determined in tumor-free athymic nude mice. Agents are prepared as suspensions in sterile water containing 0.5% methylcellulose (w/v) and 0.1% Tween 80 (v/v) and administered to mice (7 animals/group) by oral gavage at doses of 0, 25, 50, 100 and 200 mg/kg once daily for 14 days. Body weights, measured twice weekly, and direct daily observations of general health and behavior will serve as primary indicators of drug tolerance. MTD is defined as the highest dose that causes no more than 10% weight loss over the 14-day treatment period.

The maleimide derivatives can also be provided as pharmaceutically acceptable salts. The phrase “pharmaceutically acceptable salts” connotes salts commonly used to form alkali metal salts and to form addition salts of free acids or free bases. The nature of the salt is not critical, provided that it is pharmaceutically acceptable. Suitable pharmaceutically acceptable acid addition salts of the compounds may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic, and sulfonic classes of organic acids, examples of which include formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, ambonic, pamoic, methanesulfonic, ethanesulfonic, benzenesulfonic, pantothenic, 2-hydroxyethanesulfonic, toluenesulfonic, sulfanilic, cyclohexylaminosulfonic, stearic, algenic, γ-hydroxybutyric, galactaric, and galacturonic acids. Suitable pharmaceutically acceptable base addition salts of the compounds described herein include metallic salts made from aluminum, calcium, lithium, magnesium, potassium, sodium, and zinc. Alternatively, organic salts made from N,N′-dibenzylethylenediamine, chloroprocaine, choline, diethanolamine, ethylenediamine, meglumine (N-methylglucanine) and procaine may be used form base addition salts of the compounds described herein. All of these salts may be prepared by conventional means from the corresponding compounds described herein by reacting, for example, the appropriate acid or base with the compound.

### Preparation of Anticancer Compounds

Compounds of the invention may be synthesized by synthetic routes that include processes analogous to those well known in the chemical arts, particularly in light of the description contained herein. The starting materials are generally available from commercial sources such as Aldrich Chemicals (Milwaukee, Wisconsin, USA) or are readily prepared using methods well known to those skilled in the art (e.g., prepared by methods generally described in Louis F. Fieser and Mary Fieser, Reagents for Organic Synthesis, v. 1-19, Wiley, New York, (1967-1999 ed.) and similar texts known to those skilled in the art.

The present invention is illustrated by the following example. It is to be understood that the particular example, materials, amounts, and procedures are to be interpreted broadly in accordance with the scope and spirit of the invention as set forth herein.

### EXAMPLE

### Example 1: GSK-3 Inhibitor Elraglusib Enhances Tumor-Infiltrating Immune Cell Activation in Tumor Biopsies and Synergizes with Anti-PD-L1 in a Murine Model of Colorectal Cancer

We sought to evaluate elraglusib (9-ING-41), a small molecule that targets GSK-3 which has the potential to increase the efficacy of immune checkpoint blockade (ICB). We chose to evaluate elraglusib, which inhibits both α and β isoforms, because it is a clinically relevant small molecule with superior pharmacokinctic properties and is significantly more potent than other GSK-3 inhibitors (8,9). Although there are ongoing efforts to further characterize the immunomodulatory effects of GSK-3 inhibitors, few utilize small-molecule elraglusib. Rudd et al., Cell Rep., 30(7):2075-82 (2020).

Here, we characterize the effects of elraglusib in vitro on tumor and immune cells, in vivo in combination with ICB in a syngeneic murine colon carcinoma BALB/c model using MSS cell line CT-26, and in human tumor biopsies and plasma samples from patients with refractory solid tumors of multiple tissue origins enrolled in a Phase 1 clinical trial investigating elraglusib.

### Results

### Elraglusib Sensitizes Tumor Cells to Immune-Mediated Cytotoxicity

A co-culture of fluorescently-labeled SW480 MSS CRC cells and TALL-104 CD8+ T cells treated with elraglusib led to an increase in tumor cell death after 24 hours. Treatment doses chosen were significantly less than the 24- and 72-hour IC-50s calculated for all cell lines evaluated in the co-culture to ensure the majority of tumor cell death was immune cell-mediated. We observed limited tumor cell death in SW480 monocultures treated with drug only. In the co-culture with tumor and immune cells only, in the absence of the drug, we noted that the baseline percentage of dead cells out of total cells was approximately 40%, after normalization. Co-cultures of tumor cells and TALL-104 T cells treated with 5 μM elraglusib had an average of 60% dead cells, while co-cultures treated with 10 μM of elraglusib had an average of 65% dead cells.

Because TALL-104 cells are a human leukemic T cell line, we next wanted to determine the relevancy of these results using normal T cells. Donor-derived CD8+ T cells were isolated from a donor blood sample in accordance with an IRB-approved protocol. A co-culture of fluorescently-labeled SW480 tumor cells and CD8+ donor-derived CD8+ T cells was then treated with elraglusib and the percentage of dead cells out of total cells was quantified after 24 hours. We again observed limited tumor cell death in SW480 monocultures treated with drug only. The data was then normalized, as previously described, and we noted even more robust immune cell-mediated tumor cell death in the co-cultures treated with elraglusib. Co-cultures of tumor cells and donor-derived CD8+ T cells treated with 5 μM elraglusib had an average of 65% dead cells, while co-cultures treated with 10 μM of elraglusib had an average of 75% dead cells.

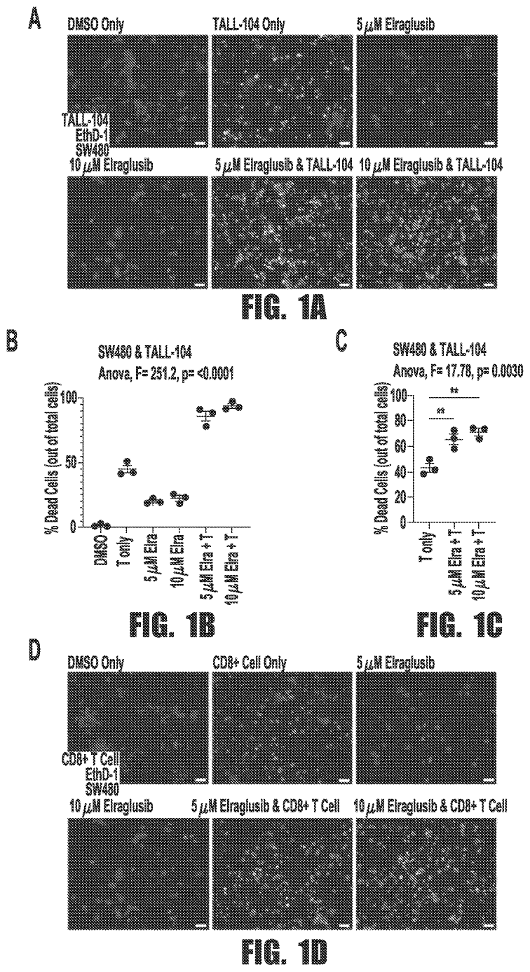

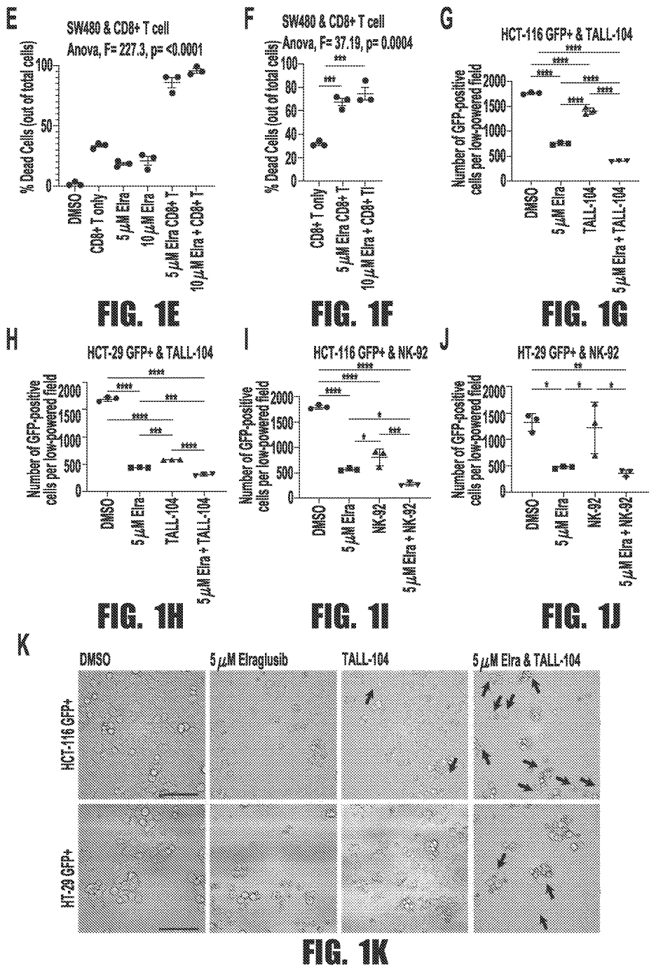

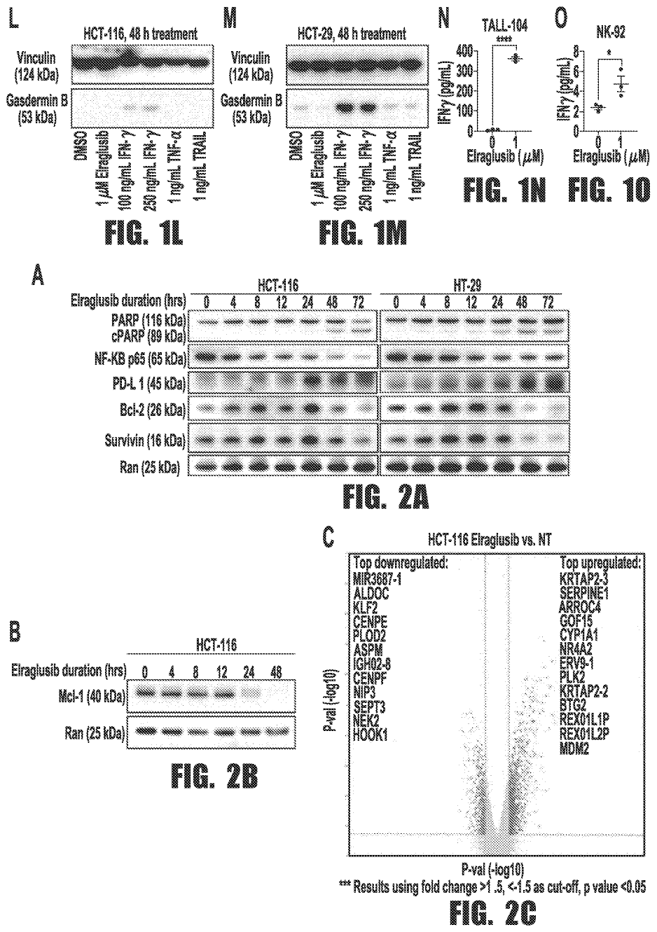

To determine if the increased amount of immune-cell mediated tumor cell-killing was due to the drug's impact on the tumor cells or the immune cells, we next pre-treated tumor cells with elraglusib for 24 hours before the co-culture with immune cells began. We observed that pre-treatment with elraglusib sensitized SW480 tumor cells to TALL-104 cell-mediated tumor cell killing (FIG. 1A). We again used the raw percentages of cell death to normalize the data and observed minimal amounts of drug cytotoxicity at the concentration and duration of treatment used (FIG. 1B). Tumor cells pre-treated with 5 μM elraglusib for 24 hours and then co-cultured with TALL-104 T cells had an average of 65% dead cells (FIG. 1C). Once again, we sought to confirm these co-culture results using donor-derived CD8+ T cells instead of TALL-104 cells (FIG. 1D). We observed similar results with the CD8+ T cells where elraglusib pre-treatment of tumor cells led to a statistically significant increase in tumor cell death after 24 hours of co-culture (FIG. 1E). Tumor cells pre-treated with 5 μM elraglusib for 24 hours and then co-cultured with donor-derived CD8+ T cells had an average of 65% dead cells, while co-cultures treated with 10 μM of elraglusib for 24 hours and then co-cultured with donor-derived CD8+ T cells had an average of 70% dead cells (FIG. 1F).

To confirm these results, we repeated these experiments using a GFP+co-culture system with additional CRC cell lines HCT-116 and HT-29. We chose to evaluate both HCT-116 and HT-29 CRC cells in this co-culture model to determine if the elraglusib-mediated increase in immune cell-mediated SW480 cell killing could be reproduced in additional CRC cell lines. These cell lines were selected based on their varied mutational profiles, with both MSI-H and MSS statuses reflected. When HCT-116 GFP+ cells were co-cultured with TALL-104 cells in the presence or absence of 5 μM elraglusib we noted a significant decrease in GFP+ cells per low-powered field in the 5 μM elraglusib only, TALL-104 only, and combination therapy groups as compared to the DMSO only control group (FIG. 1G). We noted a significant decrease in the number of GFP+ cells per field in the combination therapy group of TALL-104 and 5 μM elraglusib co-culture condition as compared to TALL-104 which recapitulated the results observed in the first co-culture system. We observed a similar trend in the HT-29 cell line where the combination therapy group showed increased tumor cell death as compared to the drug-only or T cell-only groups (FIG. 1H). To determine if these results applied to other cytotoxic immune cell lines we repeated the co-culture experiments with a natural killer cell line, NK-92. We observed similar trends in the co-culture of NK-92 cells with HCT-116 cells where the combination of 5 μM elraglusib and NK-92 cells showed increased tumor cell death as compared to the drug-only treatment or immune cell-only treatment (FIG. 1I). In the HT-29 cells, we noted increased tumor cell death in the combination therapy group as compared to immune cell only and as compared to DMSO only (FIG. 1J).

### Elraglusib Enhances Tumor Cell Pyroptosis in a Co-Culture of Colorectal Cancer Cells and Immune Cells

To determine if pyroptosis-mediated immune cell activity played a role in the co-culture results observed, we examined higher-power co-culture images for evidence of pyroptosis. Indeed, we observed some pyroptotic events in the co-cultures involving tumor cells and TALL-104 cells only (FIG. 1K). We did not observe any pyroptotic events in the DMSO or drug-only conditions suggesting that tumor cell pyroptosis was mediated by an immune cell-secreted molecule as it was only observed in the co-culture wells with immune cells (FIG. 1K). Interestingly, in the co-culture of CRC (HCT-116, HT-29) and TALL-104 cells in the presence of 5 μM elraglusib treatment, we noted a significant increase in pyroptotic events (FIG. 1K). To determine what immune cell-secreted molecules were most likely contributing to tumor cell pyroptosis, we probed for downstream mediator of pyroptotic death gasdermin B expression in tumor cells treated with a vehicle-only control (DMSO), 1 μM elraglusib, 100 mg/mL IFN-γ, 250 ng/mL IFN-γ, 1 ng/mL TNF-α, and 1 ng/mL TRAIL (FIG. 1L). We observed an increase in gasdermin B expression with both concentrations of IFN-γ used in both the HCT-116 cells and the HT-29 cells suggesting that IFN-γ secreted by immune cells was a major contributor to the pyroptosis observed (FIG. 1M). To test whether immune cells secrete more IFN-γ post-treatment with elraglusib, we treated immune cell lines (TALL-104, NK-92) with elraglusib for 24 hours and indeed noted a significant increase in IFN-γ post-treatment in cell culture supernatants (FIG. 1N-O).

### Elraglusib Upregulates Tumor Cell PD-L1 and Proapoptotic Pathway Expression as Well as Downregulates Immunosuppressive/Angiogenic Protein Expression and Pro-Survival Pathways

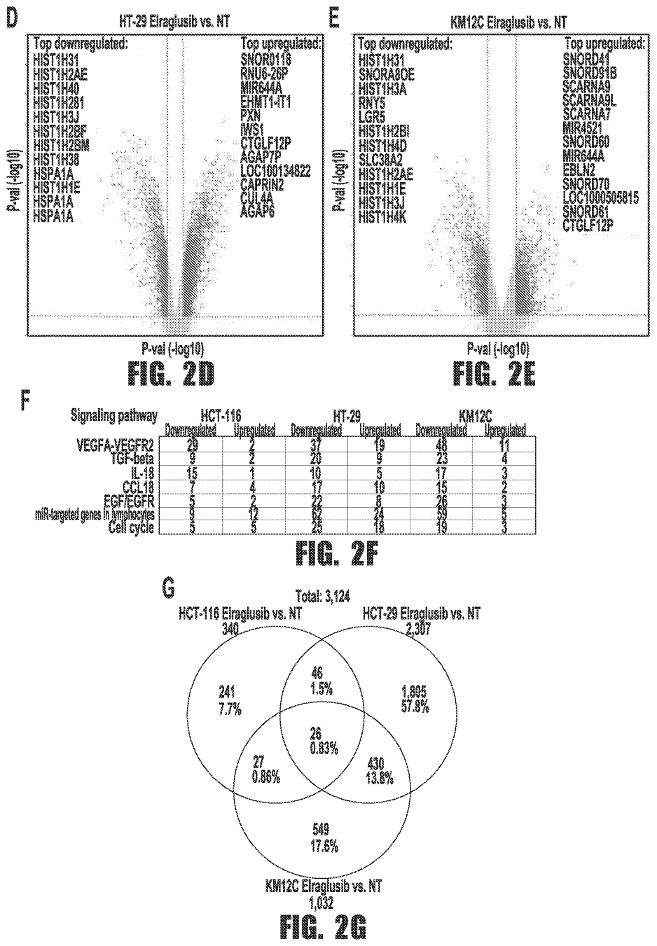

To help elucidate the mechanism behind the CRC cell sensitization to immune cell killing that we observed in the co-culture assays, we performed western blot analyses on CRC cells (HCT-116, HT-29) treated with elraglusib over a 72-hour timecourse. Using the same low dose of elraglusib utilized in the co-culture assays, we observed little to no cleaved PARP (cPARP) in both cell lines analyzed until the 48-hour timepoint confirming that the tumor cell death observed in the co-culture assays was not a product of drug cytotoxicity (FIG. 2A). Because GSK-3 is a known regulator of NF-κB signaling pathways we also probed for NF-κB p65 and noted decreased expression as the time-course progressed. However, we observed increases in PD-L1 expression as the treatment duration increased. To further elucidate elraglusib-mediated effects on tumor cell survival we probed for survival factors Bcl-2 and Survivin and noted decreases in protein expression in both cell lines analyzed, especially at the later timepoints (48, 72 hr). In HCT-116 cells, we also probed for survival factor Mcl-1 and again noted marked decreases in protein expression by the 24-hour timepoint (FIG. 2B).

We then utilized microarray analysis to gain insights into gene expression changes in CRC cell lines post-GSK-3 inhibition with elraglusib. Several CRC cell lines (HCT-116, HT-29, KM12C) were treated with elraglusib at IC-50 concentrations or DMSO as vehicle control for 24 hours and treated versus untreated samples were compared in triplicate using microarray analysis. Results were calculated using a fold change (FC) cutoff of >1.5, <−1.5, and a minimum p-value of <0.05. HCT-116 cells had 340 differentially expressed genes post-treatment (FIG. 2C). Top differentially expressed genes of interest that were upregulated in HCT-116 cells included many anti-proliferative (BTG2, TP53INP1, LYZ, GADD45A, CDKN1A, ATF3, SESN1, SUSD6) and proapoptotic (DRAM1, FAS, BLOC1S2, TNFRSF10B, KLLN, PLK3, MXD1, GADD45B, TRIM31, TP53I3, TNFRSF10A, BAK1) genes. Of note, several of the upregulated genes are known p53 targets (BTG2, MDM2, TP53INP1, DRAM1, GADD45A, CDKN1A, PMAIP1, ATF3, FAS, SESN1, TNFRSF10D, TNFRSF10B, AEN, PLK3, TP53I3, SUSD6, GDF15) (13). Meanwhile, many of the downregulated genes included those that promote cell cycle progression (CDC25C, PRC1, ANLN, BARD1, PDK1, DHX32, CCNF, PRR11, TTK, FANCD2, AURKB, UHRF1), EMT (ENO2, MST1R) or cellular proliferation (FASN, ARHGEF39, FOXC1, CDCA3, MKI67). Another upregulated (1.78-FC) gene of interest was PPP1R1C and increased expression may increase tumor cell susceptibility to TNF-induced apoptosis. Zeng et al., Biotechnol Appl Biochem., 54(4):231-8 (2009). Interestingly, CMTM4 expression was downregulated (−1.84-FC) post-treatment and is known to protect PD-L1 from being polyubiquitinated and targeted for degradation. Mezzadra et al., Nature, 549(7670):106-10 (2017). Furthermore, NEK2 was downregulated (−2.21-FC) post-treatment and NEK2 protein inhibition is known to sensitize PD-L1 blockade. Zhang et al., Nature Communications, 12(1):4536 (2021).

In HT-29 cells, we observed 2,307 differentially expressed genes post-treatment (FIG. 2D). We also observed that many of the upregulated genes post-treatment were proapoptotic (AEN, TNFRSF12A, CCAR1, SFN) or anti-proliferative (SOCS7, CDKN1A, SMAD3, BCCIP, CRLF3) and many of the downregulated genes were involved with the promotion of cellular proliferation (TNIK, BRAF, EAPP, JAK1, PDS5B, CDCA3), cell cycle progression (MCIDAS, DYNC1H1, CDC45, UHRF1, CDK2, CDC25C, CCNE1, CDK1, BARD1, CCNE2), EMT (MTA3, AGGF1, E2F8, E2F7), or have antiapoptotic functions (PIM1, SGK1, BCL6, E2F7, TRIB1). Interestingly, NCR3LG1 (B7-H6) was upregulated (1.78-FC) post-treatment and is known to trigger NCR3-dependent NK cell activation and cytotoxicity. Brandt et al., Journal of Experimental Medicine, 206(7):1495-503 (2009).

Finally, KM12C cells had 1,032 differentially expressed genes post-treatment (FIG. 2E). We observed upregulation of proapoptotic genes (TNFRSF12A, BIK) while we observed downregulation of genes involved with the promotion of EMT (CXCL1, AGGF1, IRF2BP2, MET, NRP1, GDF15, E2F8), the promotion of cell cycle progression (CDCA2, IGFBP2, CDC25C, CCNE1, CCND2, CDK1, CCNE2, BARD1), cellular proliferation (MKI67, BRAF), and the regulation of TGFβ signaling (TGFBR2, LTBP1, TGFBR3, CD109) in KM12C cells post-treatment as compared to control. Of note, we noticed an upregulation (1.53-FC) of GZMA (granzyme A) expression post-treatment which is known to cleave gasdermin B to induce pyroptosis.

Several relevant signaling pathways had differentially expressed genes post-elraglusib in all three cell lines examined including the VEGFA-VEGFR2, TGFβ, IL-18, CCL18, EGF/EGFR, miR-targeted genes in lymphocytes, Apoptosis, and cell cycle signaling pathways (FIG. 2F). The most significant commonly downregulated signaling pathway was VEGFA-VEGFR2 which had 29 downregulated genes in HCT-116 cells, 37 downregulated genes in HT-29 cells, and 48 downregulated genes in KM12C cells. A Venn Diagram was used to compare the 3,124 genes that were differentially expressed post-treatment as compared to control with elraglusib in the three colon cancer cell lines (HCT-116, HT-29, KM12C) (FIG. 2G). HCT-116 cells had 241 (7.7%), HT-29 cells had 1,805 (57.8%) and KM12C cells had 549 (17.6%) differentially expressed genes post-treatment as compared to control. HCT-116 and HT-29 cells shared 46 differentially expressed genes (1.5%), HCT-116 and KM12C shared 27 (0.86%), KM12C and HT-29 shared 430 (13.8%), and all three cell lines shared 26 (0.83%). All three cell lines showed post-treatment differential expression of NF-κB regulators with increased expression of many negative regulators of NF-κB (NFKBIA, TNFAIP3, TRAIP, IL32) and decreased expression of several positive regulators of NF-κB (IRAK1BPT, FADD, IL17RA, MYD88, ERBB2IP, IL17RB, TNFSF15, NFKBIZ, NFKBIA, MAP3K1, TRAF5, TRAF6, TAB3, TNFRSF11A, MTDH, TLR3).

We previously found that elraglusib treatment of human CRC cell lines (HCT-116, HT-29, KM12C) with varied mutational profiles modified cytokine, chemokine, and growth factor secretion into cell culture media. Huntington et al., Oncotarget, 12(20), 1980-1991 (2021). Here, we treated tumor cells (HCT-116, HT-29) with 1 μM or 5 μM elraglusib for 48 hours and subsequently analyzed the cell culture supernatant using Luminex 200 technology (FIG. 2H-I). Several cytokines, chemokines, and growth factors associated with angiogenesis and/or EMT were downregulated in both cell lines (HCT-116, HT-29) at both concentrations of elraglusib. Notably, GDF-15, GM-CSF, and VEGF all had decreased secretion post-treatment in both cell lines and at both concentrations of elraglusib. Likewise, several cytokines, chemokines, and growth factors associated with immunosuppression were also downregulated post-treatment including CCL5/RANTES, DcR3, Fas, and soluble PD-L1 (sPD-L1).

### Elraglusib Enhances Immune Cell Effector Function

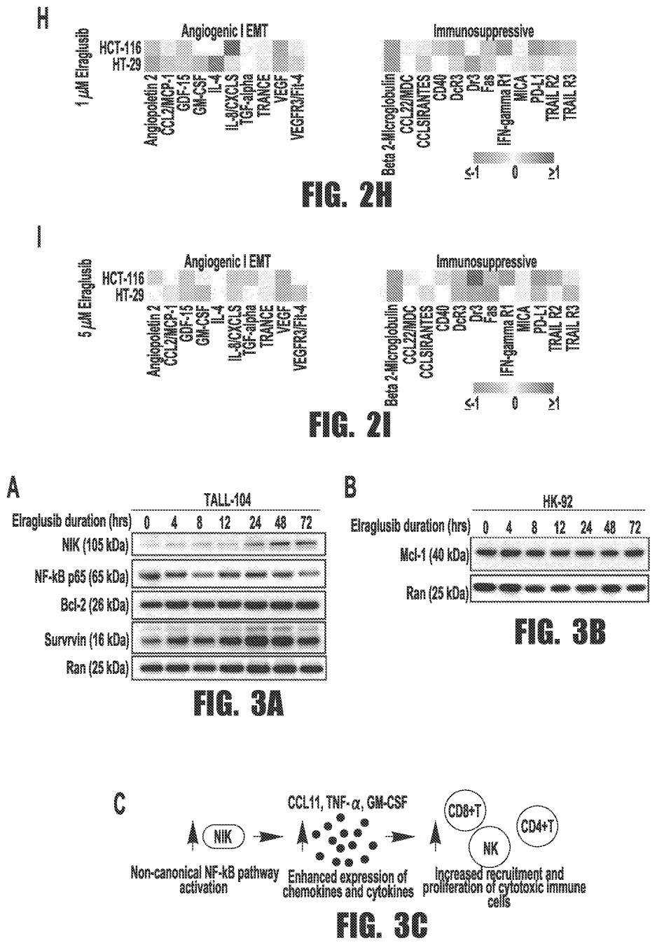

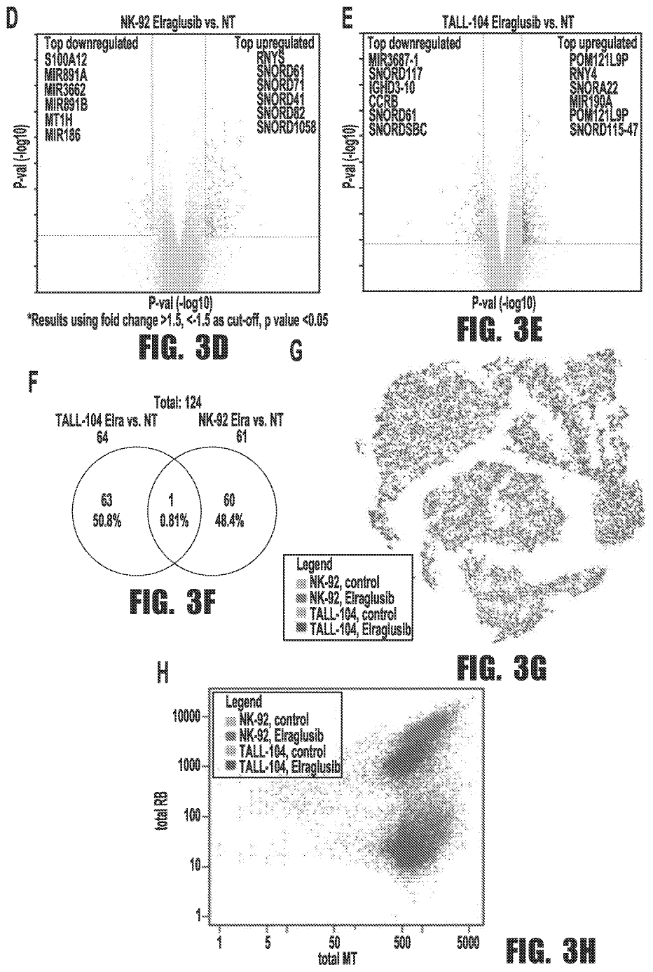

We next analyzed immune cell lines (TALL-104, NK-92) using western blot analysis. Interestingly, when we probed for the same proteins in the cytotoxic immune cell lysates, we observed many opposing trends to those observed in the tumor cells. In TALL-104 cells, we did not notice significant changes in NF-κB or survival protein Bcl-2 as treatment duration increased (FIG. 3A). Because of the differential impact of elraglusib on tumor and immune cells that we observed via western blot, we next sought to compare the levels of another survival protein Mcl-1 in NK-92 natural killer cells and we did not observe a significant decrease in Mcl-1 protein expression through the 72-hour endpoint (FIG. 3B). Surprisingly, we noted increases in survival protein Survivin and NF-κB-inducing kinase (NIK), a protein commonly associated with activation of the non-canonical NF-κB signaling pathway which led us to create a working model of NIK-mediated increased immune cell recruitment (FIG. 3C). Although GSK-3 plays a role in the regulation of D-catenin, we did not focus on elraglusib-mediated effects on β-catenin because colon cancers often harbor mutations in β-catenin or adenomatous polyposis coli (APC), thus nullifying any impact GSK-3 inhibition would have on β-catenin expression. HCT116 cells are heterozygous for β-catenin, harboring one wild-type allele and one mutant allele with inactivation of one of the residues (SER45) phosphorylated by GSK3β that is frequently mutated in tumors. Moreover, HT-29, KM12C, and SW480 cells harbor APC mutations.

Next, microarray analysis was used to gain insights into gene expression changes in immune cell lines post-GSK-3 inhibition with elraglusib. Immune cell lines (TALL-104, NK-92) were treated with elraglusib at IC-50 concentrations or DMSO as vehicle control for 24 hours, and treated versus untreated samples were compared in triplicate using microarray analysis. Results were calculated using a FC cutoff of >1.5, <−1.5, and a minimum p value of <0.05. NK-92 cells had 61 differentially expressed genes post-treatment (FIG. 3D). We observed an increase in genes that promote immune cell proliferation (TNFSF14, RAB38) and control immune cell adhesion and migration (WNK1) post-elraglusib treatment. We also noted decreases in proapoptotic genes (MIR186, S100A12) and genes that are involved in the activation of latent TGFβ to suppress immune cell function (ITGB8). TALL-104 cells had 64 differentially expressed genes post-treatment (FIG. 3E). We observed increased expression of genes involved in the modulation of NF-κB activity (RNY4, RNY5), cytotoxic granule exocytosis (STX19, VAMP8), and anti-apoptotic gene BCL2A1 (BCL2-related protein A1). We also saw an upregulation (1.56-FC) of KIF7 (kinesin family member 7) which is required for T cell development and MHC expression as well as an increased expression (1.52-FC) of CCL3 (chemokine [C—C motif] ligand 3) which is known to recruit and enhance the proliferation of CD8+ T cells. Honey K., Nature Reviews Immunology, 6(6):427 (2006). In contrast, we observed decreased expression of genes involved in TGFβ signaling pathways (ACVR1B, PTPN14) and proapoptotic genes (HSPA1A, UBE3A). We also saw a decreased expression (−1.56-FC) of inhibitory immune checkpoint PTPN3 (protein tyrosine phosphatase, non-receptor type 3). In total there were 124 differentially expressed genes post-treatment and only 1, an unnamed gene (probe set ID TC22000564.hg.1, coding), was shared between both cell lines (FIG. 3F).

To determine if there was any heterogeneity in response to drug treatment, we employed 10× single-cell sequencing analysis on both immune cell lines (TALL-104, NK-92) treated with low-dose 1 μM elraglusib or vehicle control (DMSO) for 24 hours. As expected, samples clustered by cell type when aggregate data was visualized using a t-SNE plot (FIG. 3G). Interestingly, immune cells showed differential expression of mitochondrial-encoded genes (MT) and ribosomal genes (RB) post-treatment with elraglusib suggesting a metabolic shift in line with the extensive metabolic reprogramming undergone in immune cells post-activation to support immune cell activities such as cytokine production (FIG. 3H). Several genes showed the same trends post-treatment in both cell lines (FIG. 3I). In both cell lines, we observed an increase in immune cell activation marker CD69 and a decrease in the immunosuppressive marker CHI3L1. Finally, we noted an increase in immune cell attractant CCL4 in the NK-92 cells and an increase in immune cell chemoattractant CXCR4 in the TALL-104 cells.

Because the previously observed non-canonical NF-κB pathway activation is known to enhance the expression of immune cell chemotactic chemokines and cytokines, we sought to determine how elraglusib treatment impacts the immune cell secretome. TALL-104 and NK-92 cells were treated with 1 μM elraglusib for 48 hours before cell culture supernatant was collected for cytokine profile analysis. TALL-104 cells treated with elraglusib showed increases in effector molecules IFN-γ, granzyme B, and TRAIL concentrations, as measured in picogram per milliliter (FIG. 3J). In contrast, NK-92 cells treated with elraglusib showed increases in IFN-γ and TRAIL but showed decreases in the concentration of secreted granzyme B.

Elraglusib Significantly Prolongs Survival in Combination with Anti-PD-L1 Therapy in a Syngeneic MSS CRC Murine Model

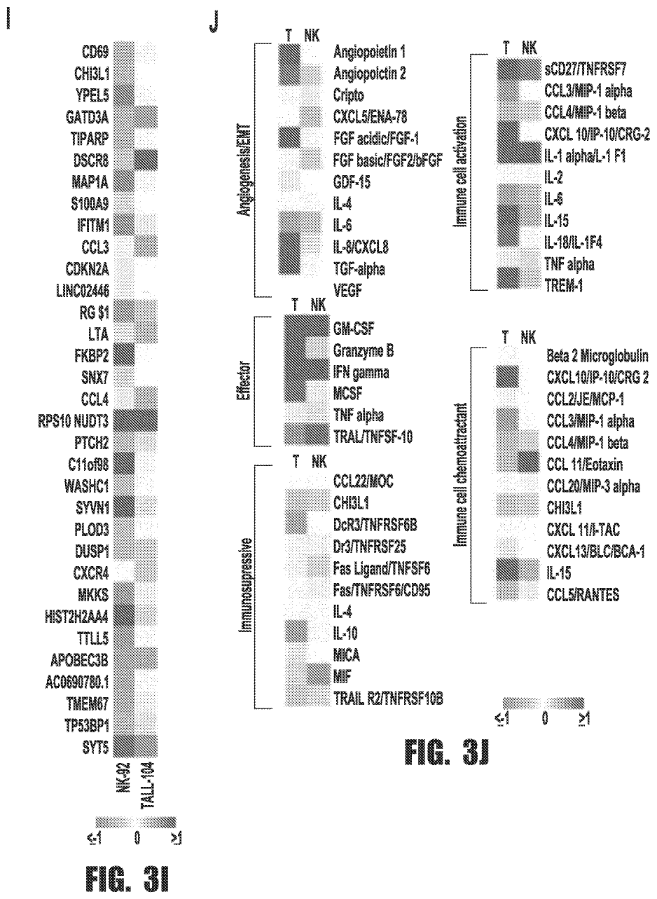

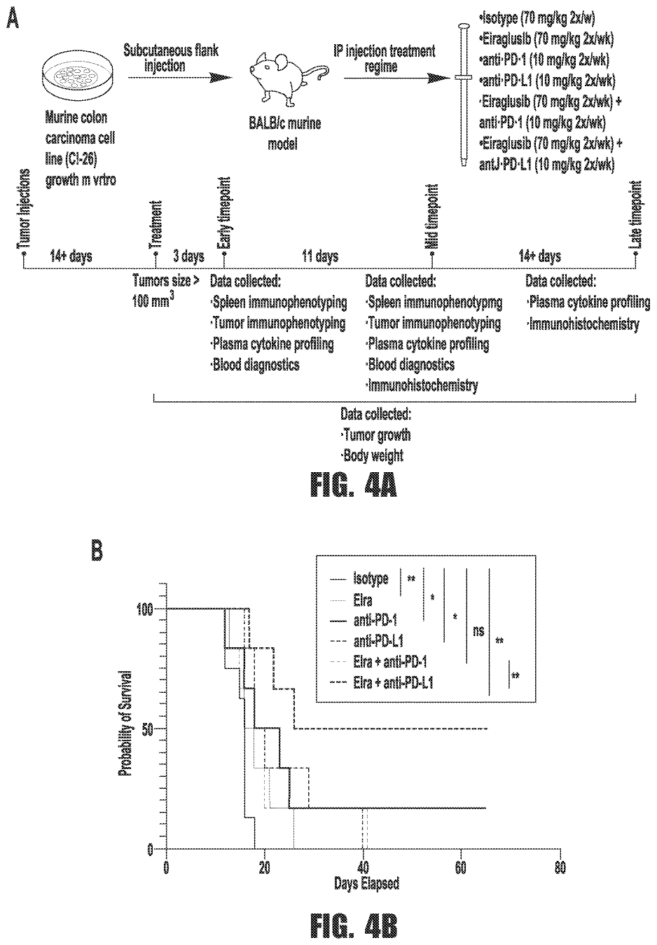

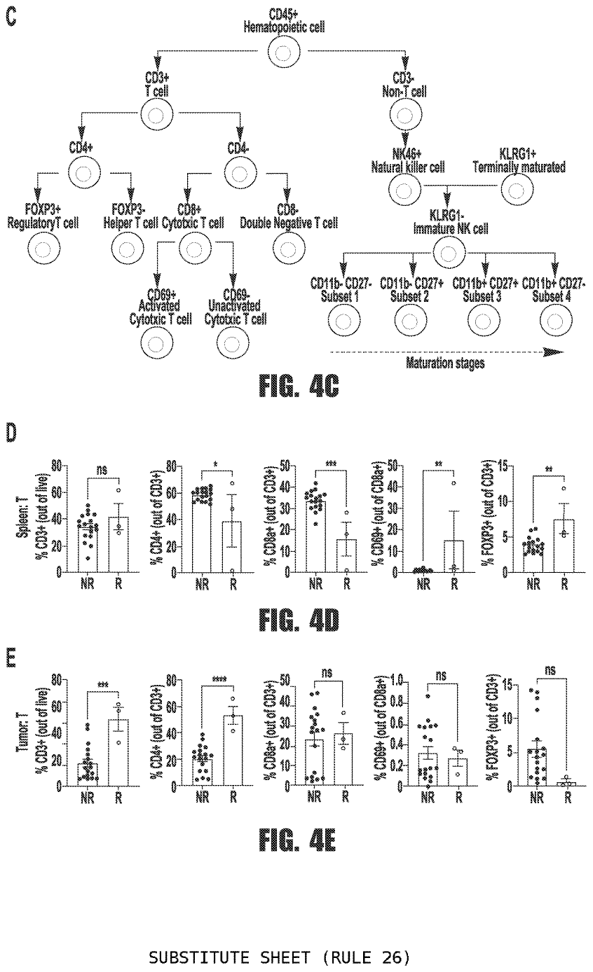

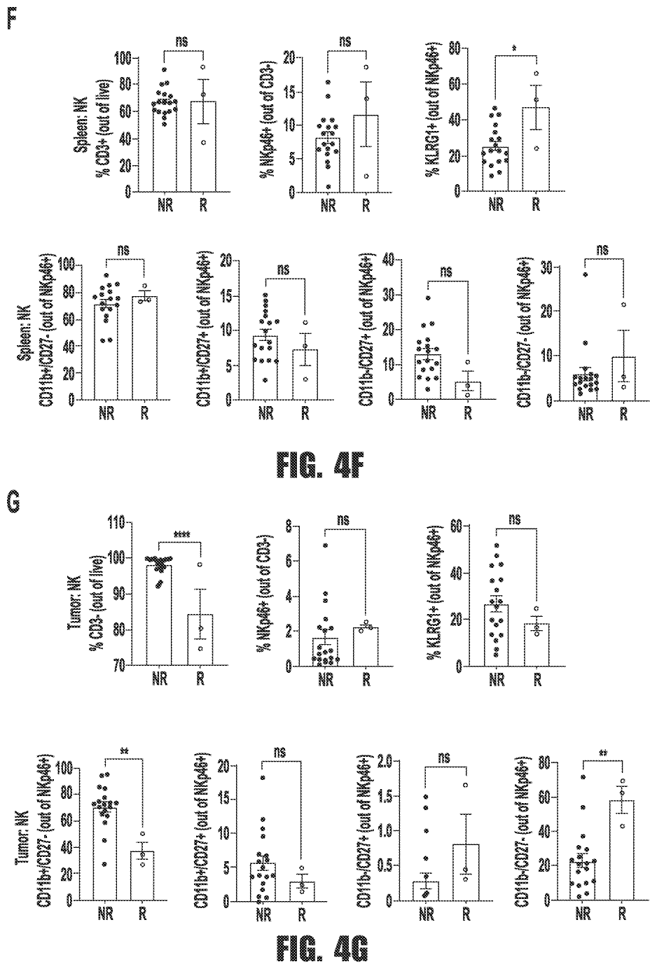

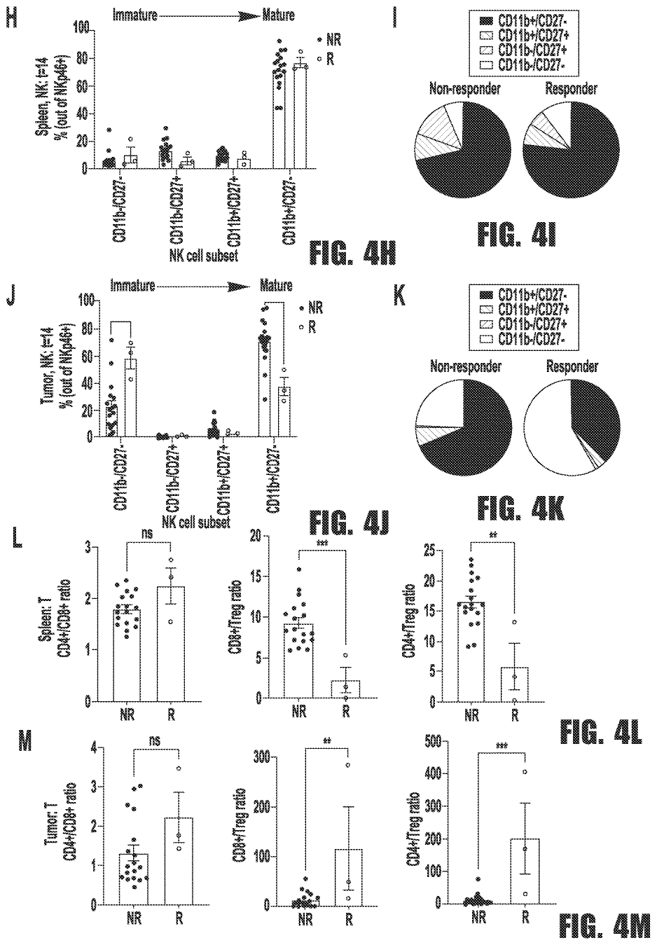

Because elraglusib activated immune cells and increased tumor cell PD-L1 expression, we sought to evaluate the potential for elraglusib to increase the efficacy of ICB and utilized a syngeneic murine colon carcinoma BALB/c murine model using a MSS cell line CT-26 (FIG. 4A). In this MSS CRC model, we observed a significantly improved survival curve in the elraglusib and anti-PD-L1 combination therapy group (FIG. 4B). We also observed statistically significant improved survival in the elraglusib, anti-PD-1, and anti-PD-L1 alone groups as compared to the control. However, we saw the most sustained response in the elraglusib and anti-PD-L1 combination therapy group. Murine body weights did not differ significantly regardless of the treatment group. Also, the mice did not show evidence of significant treatment-related toxicity on complete blood count or serum chemistry analysis. Both the elraglusib individual treatment and dual treatment groups maintained normal renal function as evidenced by normal blood urea nitrogen (BUN) and creatinine and were free of significant electrolyte perturbations. Liver function tests did not reveal any evidence of liver toxicity and the dual-treatment mice did not have any elevations of AST, ALT, or bilirubin. As can be expected in mice with significant tumor burdens, mice across treatment groups had decreased albumin levels and evidence of mild marrow hypoplasia resulting in mild anemia, and lower white blood cell and platelet counts. This effect was independent of the treatment group and likely related to tumor burden at the time of sacrifice.

Murine Responders have More T Cell Tumor-Infiltration and Higher Tumoral CD8+/Treg and CD4+/Treg Ratios

To begin to evaluate our hypothesis that elraglusib increases immune cell activation and recruitment, we utilized multi-color flow cytometry to characterize the natural killer (NK) and T cell populations 14-days post-treatment initiation, and immune cell subpopulations were analyzed in both the spleen and the tumor (FIG. 4C). 14 days post-treatment initiation, mice were grouped as responders (R) or non-responders (NR) based on a tumor volume less than or greater than 100 mm3, respectively. Compared to non-responders, regardless of treatment group, responders 14-days post-treatment had statistically significantly lower levels of splenic CD4+ and CD8+ T cells and had increased percentages of CD69+ activated T cells and Foxp3+ regulatory T cells (Tregs) (FIG. 4D). Meanwhile, responders had increased percentages of tumor-infiltrating CD3+ and CD4+ T cells (FIG. 4E). We also observed that responders had increased percentages of splenic KLRG1+ mature NK cells and tumor-infiltrating CD11b−/CD27− immature NK cells, and decreased percentages of tumor-infiltrating CD11b+/CD27− activated NK cells 14-days post-treatment initiation (FIG. 4F-G). We did not observe striking differences between non-responders and responders in the splenic immature natural killer cell subsets (CD11b−/CD27−, CD11b−/CD27+, CD11b+/CD27+, CD11b+CD27−) (FIG. 4H-I). In contrast, we did observe significant differences between non-responders and responders in the tumor-infiltrating immature natural killer cell subsets (FIG. 4J-K). We observed that responders had a greater proportion of immature (CD11b−/CD27−) NK cells and a lower proportion of mature (CD11b+CD27−) NK cells 14 days post-treatment initiation. When comparing the T cell ratios, compared to non-responders, responders had a lower splenic CD8+/Treg and CD4+/Treg ratio (FIG. 4L). The CD8+/Treg ratio is commonly used as an index of TIL's effector function (24). Additionally, responders had a higher intra-tumoral CD8+/Treg and CD4+/Treg ratio (FIG. 4M). Overall, the observed changes in immune cell subsets in responders are consistent with increased infiltration of cytotoxic immune cells into the tumor.

### Murine Responders Show an Immunostimulatory Tumor Microenvironment by IHC

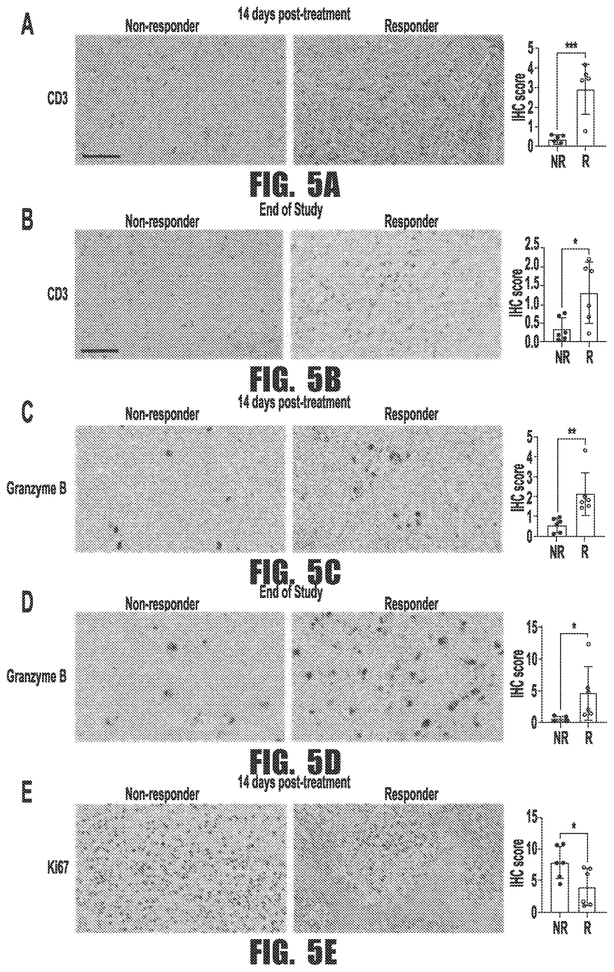

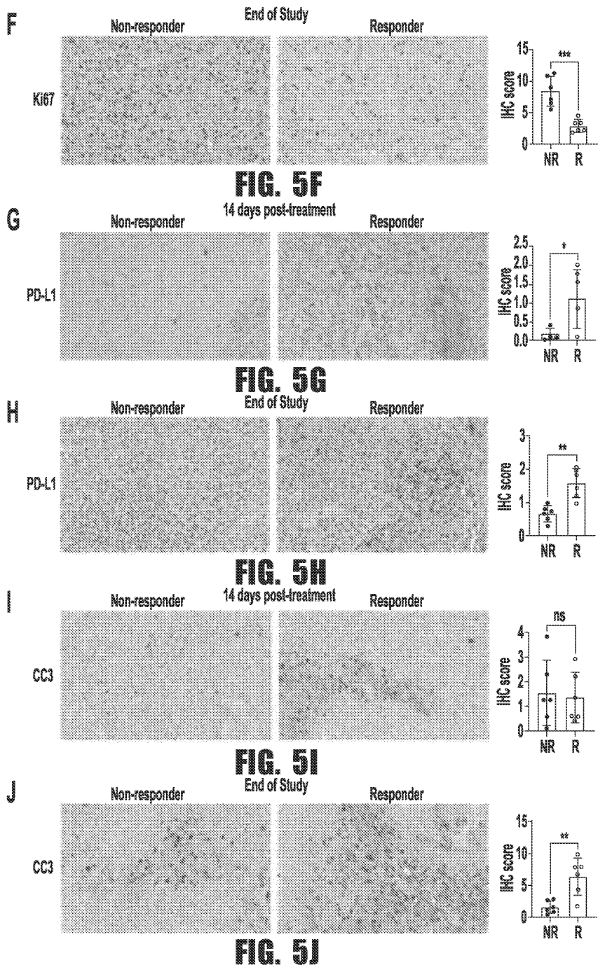

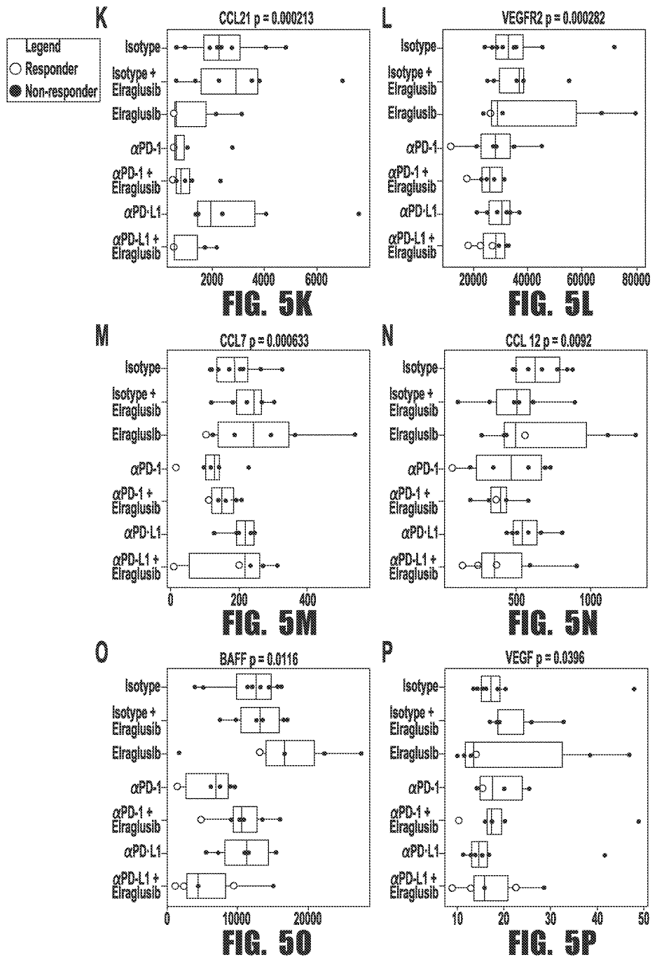

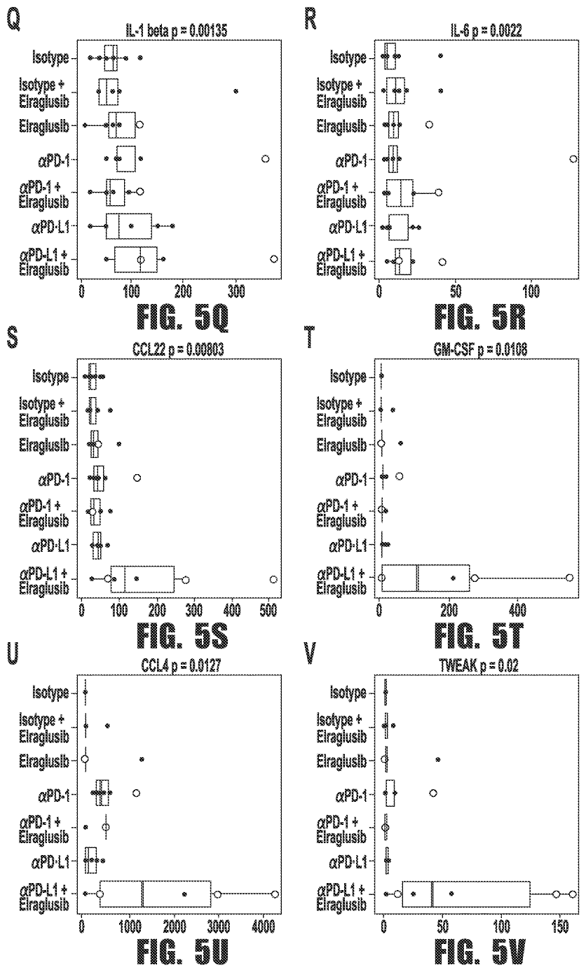

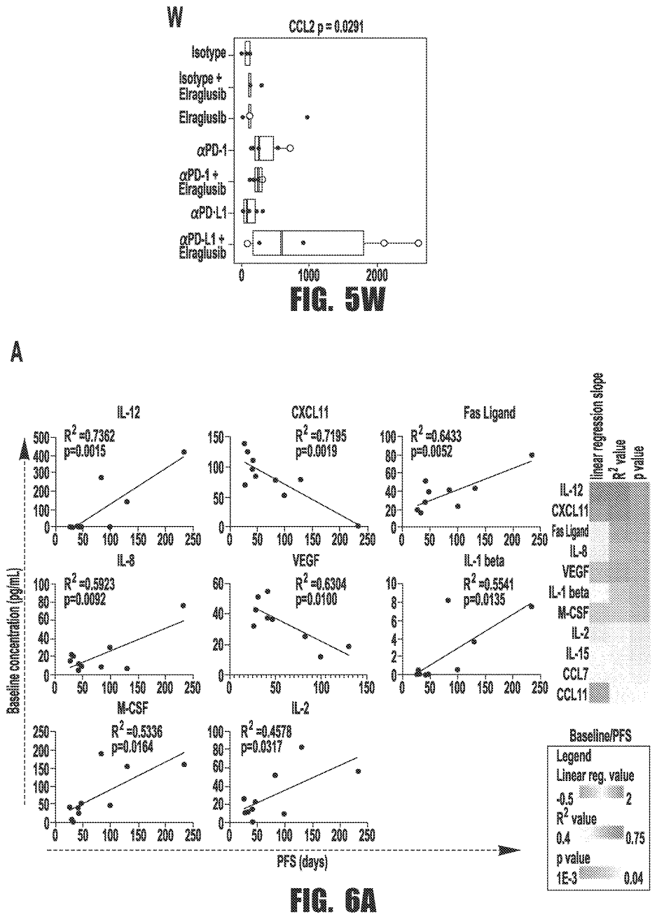

To further interrogate the tumor microenvironment (TME), we utilized immunohistochemistry (IHC) analysis on tumor sections from the 14-day post-treatment initiation timepoint or from the end-of-study (EOS) timepoint. We compared non-responders (NR) and responders (R) and stained for T cell marker CD3 and observed that responders had significantly more CD3+ T cells as compared to non-responders at both timepoints analyzed. (FIG. 5A-B). To determine if there were any differences in immune cell activation, we stained for Granzyme B and again observed that responders had significantly more Granzyme B+ staining at both timepoints analyzed as compared to non-responders (FIG. 5C-D). We stained for Ki67 as a marker of tumor cell proliferation and observed that responders had less tumor cell proliferation as compared to responders at both of the timepoints analyzed (FIG. 5E-F). Because we found that elraglusib upregulated tumor cell PD-L1 expression and because we observed such a striking difference in survival when elraglusib was combined with anti-PD-L1 therapy as compared to anti-PD-1 therapy, we next looked at PD-L1 staining in the tumor sections (FIG. 5G-H). We observed that responders had more PD-L1+ tumor cells as compared to non-responders at both timepoints analyzed. To examine tumor cell apoptosis, we then stained for cleaved-caspase 3 (CC3) and noted that there was no difference in CC3 expression at the mid-timepoint (14 days post-treatment initiation), however, responders did have significantly more CC3 expression than non-responders at the EOS timepoint (FIG. 51-J). We also analyzed the expression of CD4, CD8, Foxp3+, NKp46, TRAIL, PD-1, VEGF, and TGFβ2 to gain additional insights into the tumor immune microenvironment at both the 14 days post-treatment initiation timepoint and the EOS timepoint, respectively. To examine helper T cell presence, we stained for CD4 and observed that responders had more CD4+ T cells than non-responders at both of the timepoints examined. Interestingly, we saw the same trends when we examined CD8 expression where responders had more CD8+ T cells as compared to non-responders which differed from the flow cytometry results but could be explained by the large variability in CD8a+ T cells we observed by flow cytometry in the non-responder group. We did not observe statistically significant differences in Foxp3+ Treg expression between responders and non-responders at either timepoint. When we examined NK cell tumor-infiltration by IHC we noted more NKp46+ NK cells in responders at the 14-day post-treatment initiation timepoint, but this difference was not significant at the EOS timepoint. We chose to examine another cytotoxic mediator TRAIL, and observed no difference between responders and non-responders at the mid-timepoint but, interestingly, observed less TRAIL expression in the responders as compared to the non-responders at the EOS timepoint. We also examined PD-1 expression and did not note any significant differences between responders and non-responders at either of the timepoints examined. Again, we noted a similar lack of significance when we examined immunosuppressive and angiogenic VEGF expression. Finally, we examined immunosuppressive TGFβ2 expression and noted no differences between responders and non-responders at the mid-timepoint but noted that responders had significantly lower expression at the EOS timepoint. Signal was quantified by converting randomly sampled 20× images into 16-bit images and then utilizing Fiji to employ MaxEntropy thresholding.

Murine Responders have Lower Tumorigenic and Higher Immunomodulatory Cytokine Concentrations

We next analyzed murine serum samples from EOS mice for cytokine profiles and noted interesting trends between responders and non-responders. Responders were more likely to have lower serum concentrations of CCL21 (p=0.000213), VEGFR2 (p=0.000282), CCL7 (p=0.000633), CCL12 (p=0.0092), BAFF (p=0.0116), and VEGF (p=0.0396) compared to non-responders (FIG. 5K-P). In contrast, responders had higher serum concentrations of IL-1 f (p=0.00135), IL-6 (p=0.0022), CCL22 (p=0.00803), GM-CSF (p=0.0108), CCL4 (p=0.0127), TWEAK (p=0.02), and CCL2 (p=0.0291) compared to non-responders (FIG. 5Q-W).

Analytes that were statistically significant between responders and non-responders at both timepoints (14 days post-treatment initiation, EOS) included CCL7/MCP-3/MARC (p=2.19E-05), CCL12/MCP-5 (p=0.000606), TWEAK/TNFSF12 (p=0.00112), BAFF/TNFSF13B (p=0.00469), IL-1β/IL-1F2 (p=0.00507), CCL21/6Ckine (p=0.00539), VEGF (p=0.00646), IFN-γ (p=0.00817), CCL4/MIP-1β(p=0.0133), IL-6 (p=0.229), and GM-CSF (p=0.0257). When comparing responders and non-responders, a Kruskal-Wallis test was used to calculate statistical significance followed by a Benjamini-Hochberg correction for multiple comparisons. The entire panel of cytokines, chemokines, and growth factors analyzed by multiplex immunoassay in murine serum from the EOS timepoint included BAFF, MCP-1, MIP-1 α, MIP-1 β, RANTES, MCP-3, Eotaxin, MCP-5, VEGFR2, MIP-3 α, CCL21, MDC, IP-10, CXCL12, GM-CSF, Granzyme B, IFN-γ, IL-1 α, IL-18, IL-2, IL-3, IL-4, IL-6, IL-7, IL-10, IL-12 p70, IL-13, IL-16, VEGF, M-CSF, Prolactin, and TWEAK.

Patient Plasma Concentrations of Cytokines from a Phase 1 Clinical Trial Investigating Elraglusib Correlate with Progression-Free Survival, Overall Survival, and In Vivo Response to Therapy Results

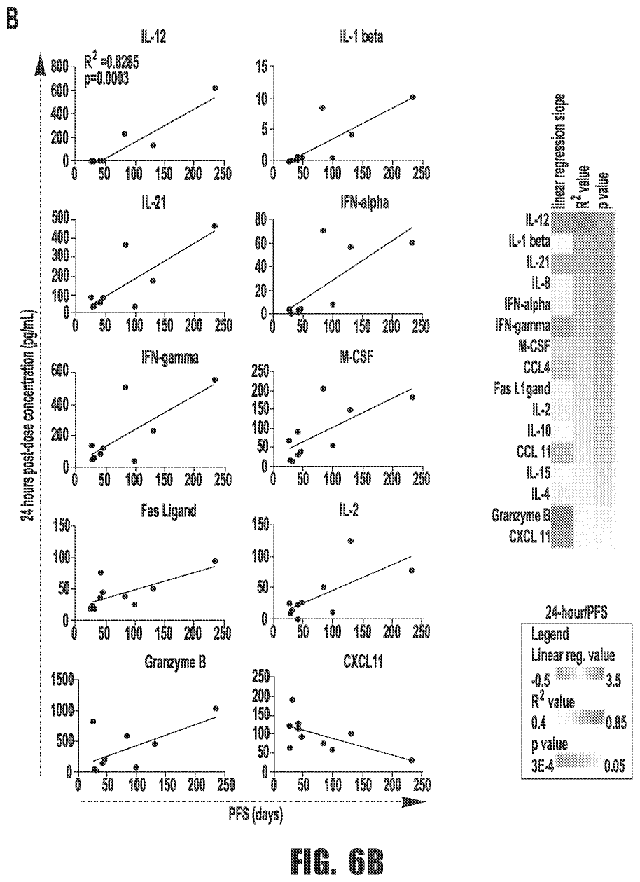

To determine the clinical relevance of the biomarkers of response identified in our murine model, we next employed Luminex 200 technology to analyze plasma samples from patients with refractory solid tumors of multiple tissue origins enrolled in a Phase 1 clinical trial investigating elraglusib (NCT03678883). Patients included in this analysis represented multiple tumor types including appendix (n=3, 15.8%), adult T-cell leukemia/lymphoma (ATLL) (n=1, 5.3%), cholangiocarcinoma (n=1, 5.3%), colorectal (n=7, 36.8%), desmoid (n=1, 5.3%), hepatocellular carcinoma (HCC) (n=1, 5.3%), leiomyosarcoma (n=1, 5.3%), non-small cell lung cancer (NSCLC) (n=2, 10.5%), and pancreas (n=2, 10.5%) cancer (FIG. 6A). The median PFS was 75.9 days, and the median OS was 101 days. Baseline and 24-hour post-elraglusib plasma concentrations of cytokines, chemokines, and growth factors were plotted against PFS and OS and simple linear regressions were used to calculate significance. A heatmap was then used to visualize linear regression values (FIG. 6B). We found that baseline concentrations of several analytes (IL-12, CXCL11, Fas Ligand, IL-8, VEGF, IL-1 β, M-CSF, IL-2) correlated with progression-free survival (PFS). Likewise, concentrations of several analytes (IL-12, IL-1β, IL-21, IL-8, IFN-α, IFN-γ, M-CSF, CCL4, Fas Ligand, IL-2, IL-10, CCL11, IL-15, IL-4, Granzyme B, CXCL11) 24-hours post-dose also correlated with PFS. We next analyzed overall survival (OS) data and noted that baseline concentrations of several analytes (IL-8, CXCL11, CCL11, IFN-α, TNF-α, Fas Ligand, TRAIL R2, IL-1β) correlated with OS. Next, 24-hour post-dose concentrations of several analytes (IFN-α, Fas Ligand, TRAIL R2, CCL11) also correlated with OS. Importantly, Fas Ligand and CCL11 were the only two analytes that were statistically significant in all four comparisons (pre-dose, post-dose, OS, PFS) and were found to be positivity correlated with improved PFS and OS.

Many of the analytes were upregulated at 8- and 24-hours post-dose as compared to baseline (FIG. 6C). When the cytokines, chemokines, and growth factors were grouped by timepoint and raw values were visualized with a heatmap we noticed several interesting trends. When grouped by primary tumor location (appendix, adult T cell leukemia/lymphoma [ATLL], cholangiocarcinoma, colorectal, desmoid, hepatocellular carcinoma [HCC], leiomyosarcoma, non-small cell lung cancer [NSCLC], pancreatic), we noted that the patient with a desmoid tumor had elevated expression of many of the analytes included in the panel. When cytokines were grouped by elraglusib dose (1, 2, 3.3, 5, 7, 9.3, 12.37) in milligrams per kilogram, we noted that patients receiving a 7 mg/kg dose had increased expression of many of the analytes included in the panel at both the 8- and 24-hour post-dose timepoints. Finally, when cytokines were grouped by cytokine, chemokine, or growth factor family we noted that TNF family molecules (BAFF, Fas Ligand, Fas, TNF-α, TRAIL R2, TRAIL R3, TRAIL, TRANCE) has a decreased expression at the 8-hour post-dose timepoint as compared to baseline and had increased over baseline levels by the 24-hour timepoint.

To compare both murine and human circulating biomarker trends, we created a table to visualize major trends (FIG. 6D). EOS analyte concentrations that positively correlated with OS in the mouse model included IL-1 β, CCL22, CCL4, TWEAK, GM-CSF, and IL-6. Those that negatively correlated with OS in the mouse model included CCL21, VEGFR2, CCL12, BAFF, and VEGF. Interestingly, we observed that many of these trends held true when analyzing the human data. IL-1 β, CCL22, and CCL4 all were positively correlated with PFS and OS in the human cohort, likewise, BAFF and VEGF were negatively correlated with OS and PFS. GM-CSF and IL-6 had opposing correlations in the human cohort as compared to the murine cohort.

PanCK+ Expression of Immunosuppressive CD39 Negatively Correlated with Time-On-Treatment (Tx Time) while CD45+ Expression of Monocyte/Macrophage Marker CD163 Positively Correlated with Tx Time

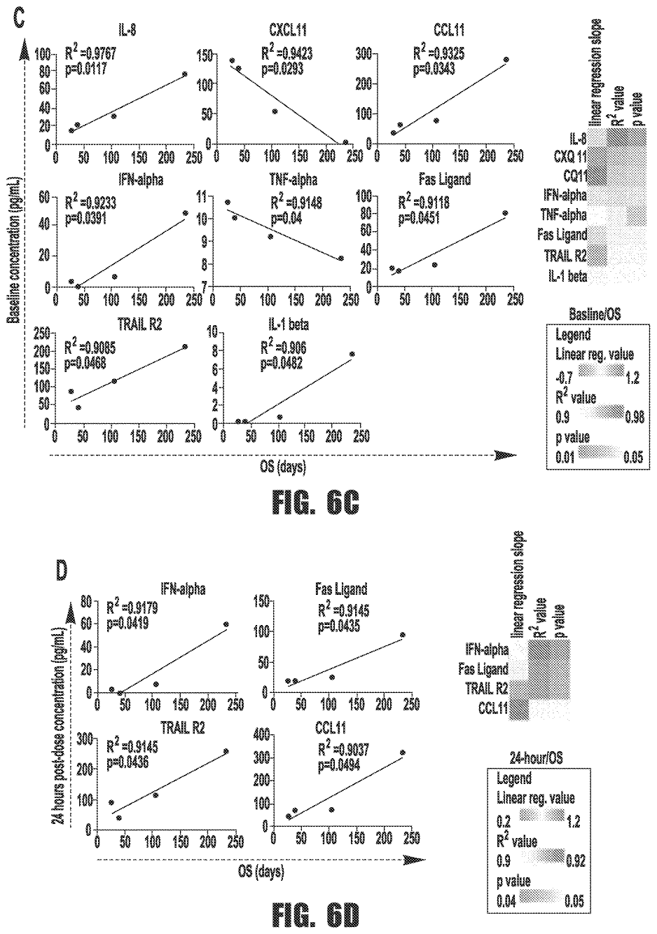

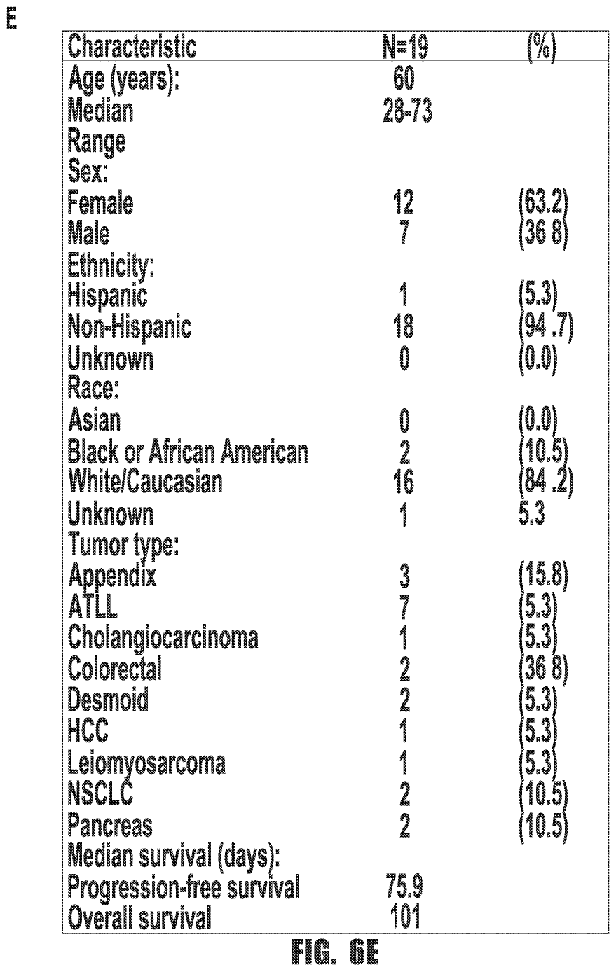

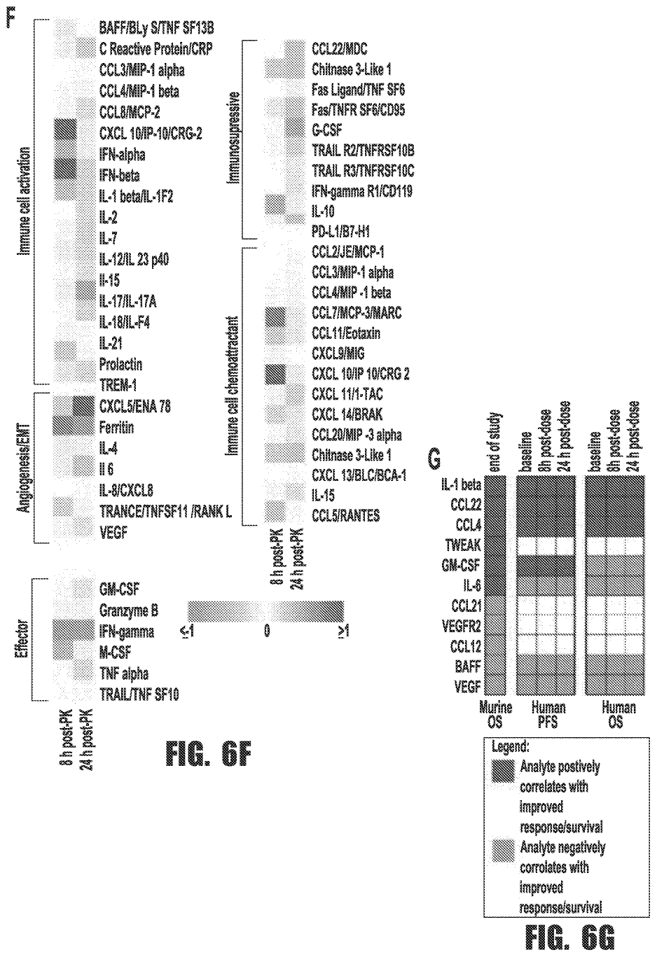

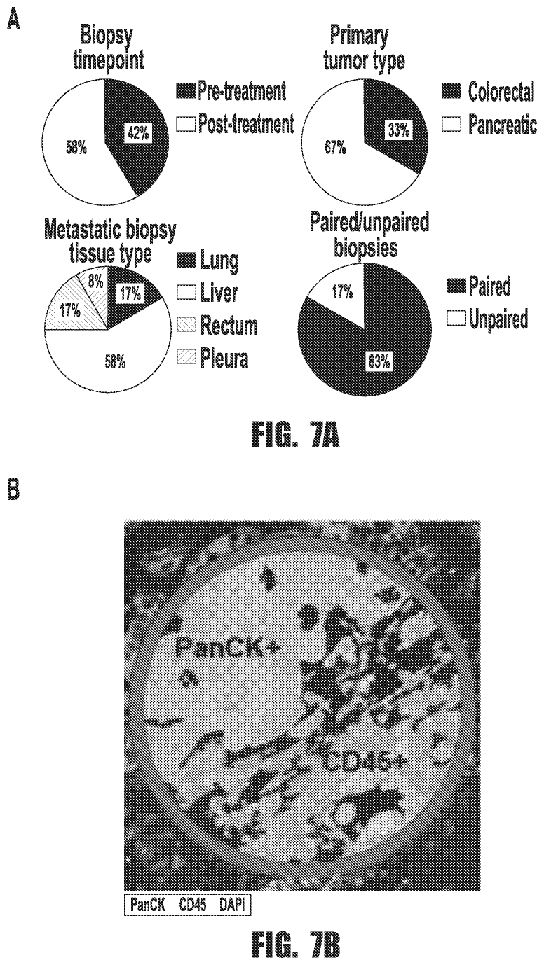

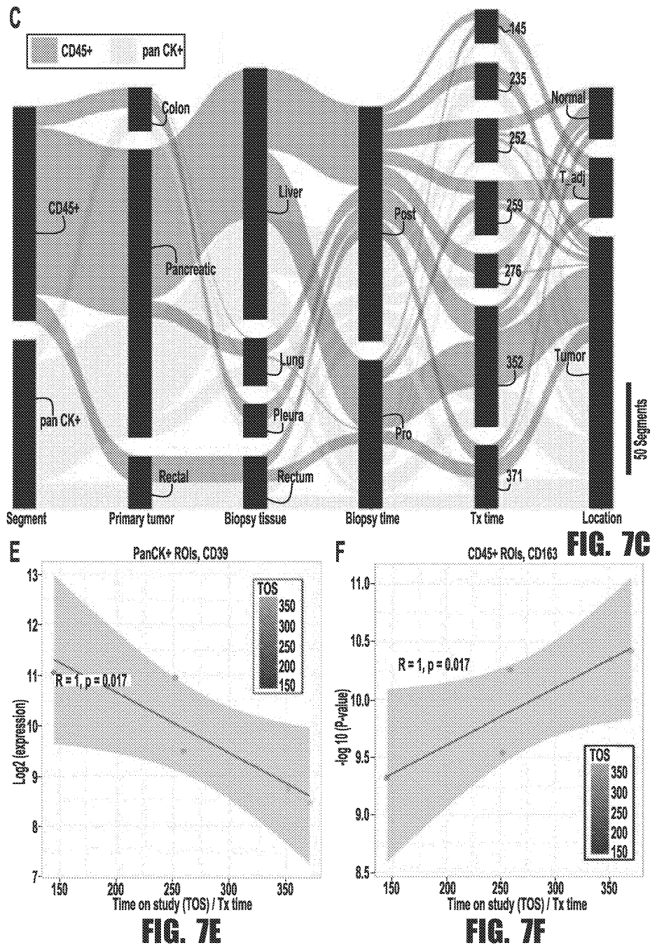

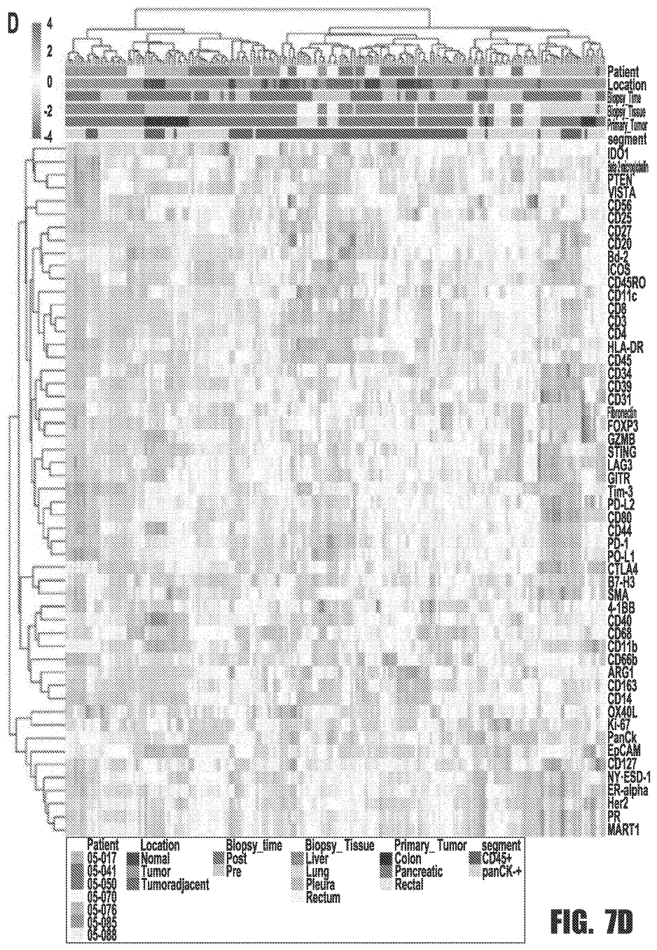

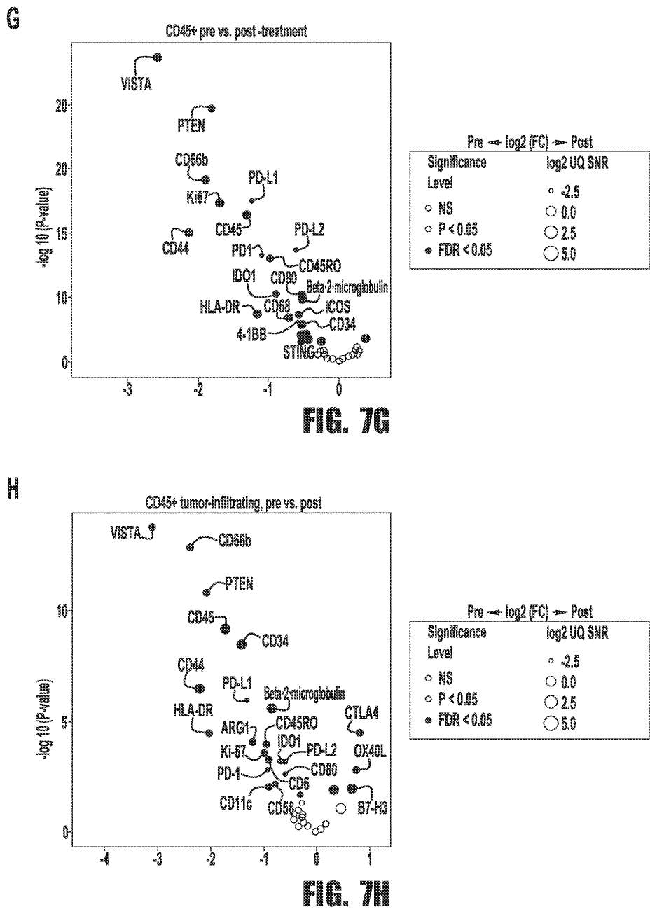

To gain insights into the human TME post-elraglusib, we utilized GeoMx Digital Spatial Profiling (DSP) technology to profile the expression of 59 proteins in tumor biopsies (n=12) from patients treated with elraglusib (n=7). 42% (n=5) of the tumor biopsies analyzed were collected near or before treatment start (pre-treatment) and 58% (n=7) of the biopsies analyzed were collected from post-treatment (average time-on-treatment [Tx time] at post-treatment biopsy: 270 days) (FIG. 7A). Primary tumor types included CRC (n=4, 33%) and pancreatic cancer (n=8, 67%), while metastatic biopsy tissue sites included lung (n=2, 17%), liver (n=7, 58%), rectum (n=2, 17%), and pleura (n=1, 8%). We analyzed five paired tumor biopsies (n=10 slides total, 83%), and 2 unpaired biopsies (n=2 slides total, 17%). Half (n=6, 50%) of the tumor sections were needle biopsies and half (n=6, 50%) were tissue biopsies. All patients included in this analysis were considered responders based on the definition used in the Phase 1 trial that treatment response is equal to disease control greater than 16 weeks. Our region of interest (ROI) selection strategy focused on mixed tumor and immune cell segments within FFPE tissue. ROIs were segmented based on panCK+ and CD45+ morphology stains to compare tumor versus immune cells protein expression (FIG. 7B). We utilized a PCA plot to visualize dimensionality reduction and as expected, samples tended to cluster by tissue type (liver, lung, pleura, rectum) and further separated by segment (CD45, panCK) on PC2. We utilized a Sankey diagram to visualize the study design where the width of a cord in the figure represents how many segments are in common between the two annotations they connect (FIG. 7C). Also as expected, samples tended to cluster together based on patient ID, primary tumor location, biopsy timepoint, metastatic biopsy tissue site, immune cell location, or segment (CD45, panCK) type when visualized on an aggregate heatmap (FIG. 7D). As we were interested in the ability to predict a patient's time-on-treatment (Tx time), we sought to correlate pre-treatment protein expression levels among the responders with Tx time data and found that PanCK+ segment expression of immunosuppressive CD39 negatively correlated with Tx time (FIG. 7E) while CD45+ segment monocyte/macrophage marker CD163 expression positively correlated with Tx time (FIG. 7F).

Tumor-Infiltrating Immune Cells have Reduced Inhibitory Checkpoint Expression and Increased Expression of T Cell Activation Markers Post-Elraglusib

When comparing all samples, CD45+ regions of post-treatment biopsies had increased protein expression of T cell activation marker OX40L (p=0.016) and decreased protein expression of checkpoint molecules VISTA (p=2.0E-24), PD-L1 (p=3.2E-13), PD-L2 (p=2.0E-9), LAG3 (p=5.1E-4), and PD-1 (p=5.6E-9). CD45+ regions of post-treatment biopsies also had decreased protein expression of myeloid/neutrophil marker CD66b (p=7.5E-15), myeloid markers IDO1 (p=4.8E-6), CD80 (p=5.4E-6), and CD11b (p=6.7E-3), TAM/M2 macrophage marker CD68 (p=3.8E-4), myeloid/T cell activation marker OX40L (p=0.016), myeloid marker CD40 (p=0.020), and DC/myeloid marker CD11c (p=0.022) as compared to pre-treatment samples (FIG. 7G). Because we were interested in the differential expression of proteins based on immune cell location in relation to the tumor, we annotated CD45+ ROI locations as tumor-infiltrating, tumor-adjacent, or normal tissue. When next compared tumor-infiltrating CD45+ immune cell segments in pre-versus post-treatment biopsies and found that post-treatment tumor-infiltrating CD45+ immune cell segments had lower protein expression of immune checkpoints VISTA (p=1.6E-14), PD-L1 (p=1.1E-6), PD-L2 (p=7.6E-4), and PD-1 (p=1.6E-3) and higher protein expression of T cell activation markers CTLA4 (p=3.1E-5) and OX40L (p=1.6E-3) (FIG. 7H). We also noted that post-treatment tumor-infiltrating CD45+ immune cell segments had lower protein expression of myeloid marker CD66b (p=1.4E-13), antigen PTEN (p=1.5E-11), hematopoietic marker CD34 (p=3.3E-9), T cell activation marker CD44 (p=3.1E-7), antigen presentation B2M (p=2.5E-6), immune cell activation marker HLA-DR (p=3.1E-5), TAM/M2 macrophage marker ARG1 (p=7.6E-5), memory T cell marker CD45RO (p=1.1E-4), proliferation marker Ki-67 (p=2.7E-4), TAM/M2 macrophage marker CD68 (p=5.8E-4), myeloid marker IDO1 (p=6.6E-4), myeloid marker CD80 (p=2.4E-3), NK cell marker CD56 (p=6.9E-3), DC/myeloid marker CD11c (p=9.5E-3), and T cell activation marker GITR (p=0.021) and had higher protein expression of immune checkpoint molecule B7-H3 (p=0.012) and Treg marker CD127 (p=0.012) as compared to pre-treatment tumor-infiltrating CD45+ immune cell segments.

Patients with a Long Time-On-Treatment have Decreased B Cell and Myeloid Marker Expression in Immune Cell Regions and have Decreased Immune Checkpoint Expression in Tumor Cell Regions

Next, we sought to compare pre-treatment biopsy protein expression in CD45+ segments between patients who were on treatment for a longer duration of time called “Long Tx patients” and patients who were on the study for a shorter duration of time called “Short Tx patients” and observed that Long Tx patients had lower protein expression of B cell marker CD20 (p=0.012) and myeloid activation marker CD80 (p=0.047). Long Tx was defined as a Tx time greater than 275 days (~39 weeks). Then we compared protein expression in CD45+ segments in post-treatment biopsies between Long Tx patients and Short Tx patients. We observed that Long Tx patients had lower protein expression of antigen NY-ESO-1 (p=0.021) and progesterone receptor (PR) (p=0.022). We then compared pre-treatment biopsy protein expression in PanCK+ segments between Long Tx patients and Short Tx patients and observed that Long Tx patients had lower protein expression of cytotoxic T cell marker CD8 (p=3.5E-3), antigen Her2 (p=0.033), Treg marker Foxp3 (p=0.033), T cell marker CD3 (p=0.035), and B cell marker CD20 (p=0.046). Long Tx patients also had lower immune checkpoint protein expression of LAG3 (p=0.023), PD-L2 (p=0.028), and PD-1 (p=0.046). We carried out the same analysis with a focus on panCK+ segments in post-treatment biopsies. Long Tx patients had lower protein expression of mature B cell/DC marker CD35 (p=8.5E-3), antigen NY-ESO-1 (p=8.7E-3), antigen Her2 (p=0.022), antigen MART1 (p=0.029), cytotoxic T cell marker CD8 (p=0.030), Treg marker Foxp3 (p=0.030), antigen PTEN (p=0.032), DC/myeloid marker CD11c (p=0.034), memory T cell marker CD45RO (p=0.036), checkpoint PD-L1 (p=0.047), and PR (p=0.049) as compared to Short Tx patients. Several additional comparisons were made between pre- and post-treatment biopsies, immune cell location in proximity to the tumor, and paired and unpaired biopsies.

### Discussion

ICB is a promising treatment strategy for many cancer patients, including MSI-H CRC patients. However, the response rate to ICB in MSS CRC patients is very limited, especially as the tumor stage advances, thus there is a clear need for improved treatment strategies for this patient population. Evaluating the combination of ICB with small molecules in oncology represents one of the ways we might improve the efficacy of ICB in MSS CRC patients. Here, we focus on small-molecule inhibitor of GSK-3 elraglusib and characterize several immunomodulatory mechanisms that provide a clinical rationale for the combination of GSK-3 inhibitors such as elraglusib in combination with ICB.

We demonstrate that small-molecule inhibition of GSK-3 using elraglusib leads to increased natural killer and T cell-mediated CRC cell killing in a co-culture model. Moreover, elraglusib acts on tumor cells to sensitize them to immune cell-mediated killing. This tumor cell sensitization could be resultant of drug-induced modifications in the tumor cell secretome such as decreased VEGF expression, decreased soluble PD-L1, and increased CXCL14, as we previously described. VEGF has been shown to inhibit T cell activation (Gavalas et al., British Journal of Cancer, 107(11):1869-75 (2012)) while CXCL14 is a known NK cell chemoattractant. Starnes et al., Experimental Hematology, 34(8):1101-5 (2006) It has been shown that the soluble or shed version of PD-L1 can retain the ability to bind PD-1 and function as a decoy receptor to negatively regulate T cell function, despite being a truncated version lacking the membrane domain of the protein. Therefore, the increase in efficacy in combination with ICB that we observed in the co-culture model could be due to a concomitant downregulation of sPD-L1 and an upregulation of cell surface-expressed PD-L1.

Elraglusib-mediated immunostimulation may also function, in part, by inducing pyroptosis in cancer cells. Pyroptosis is a lytic and pro-inflammatory type of programmed cell death that results in cell swelling and membrane perforation. Although the role of pyroptosis in cancer is controversial, it has been suggested that pyroptosis may contribute to anti-tumor immunity. Lu et al., Cancers (Basel). 2021. PMID: 34298833. Since we observed gasdermin B expression post-IFN-γ treatment in CRC cells and because we found that elraglusib treatment upregulated immune cell IFN-γ secretion, we hypothesize that the IFN-γ released from CD8+ T cells and NK cells is responsible for triggering pyroptosis which may, in part, contribute to elraglusib-mediated immunostimulation.

Another mechanism behind elraglusib-mediated immunomodulation is the suppression of inflammatory NF-κB signaling and survival pathways in the tumor cells. We demonstrated that elraglusib treatment of CRC cells decreased Survivin, NF-κB p65, Bcl-2, and Mcl-1 expression while increasing PD-L1 expression. This is in accordance with previous studies that have shown that GSK-3 is a positive regulator of NF-κB. Medunjanin et al., Sci Rep., 6:38553 (2016). Microarray data showed increased expression of antiproliferative, proapoptotic, and NF-κB regulator genes and decreased expression of genes involved in cell cycle progression, antiapoptotic, and EMT genes in CRC cell lines. Multiplex immunoassay data showed decreased tumor cell secretion of proteins involved in angiogenesis, EMT, and immunosuppression.

Meanwhile, we observed the opposite effect on NF-κB signaling in immune cells, where we observed that drug treatment increased NF-κB-inducing kinase (NIK) expression. NIK is the upstream kinase that regulates activation of the non-canonical NF-κB signaling pathway, and may implicate a role for non-canonical NF-κB signaling in immune cells post-treatment with elraglusib, which future studies could further evaluate. It is known that increased expression of NIK leads to enhanced expression of chemokines and cytokines such as CCL3, TNF-α, and MCP-1, which thus leads to increased recruitment and proliferation of cytotoxic immune cells. Moreover, elraglusib treatment of immune cells increased effector molecule secretion in both T and NK cells as well as led to increased expression of genes involved in cytotoxic granule exocytosis, cellular proliferation, and modulators of NF-κB activity. Moreover, elraglusib treatment resulted in decreased gene expression of proapoptotic molecules and regulators of TGFβ signaling which may also contribute to the tumor suppressive and anti-angiogenic effects of elraglusib that have been previously described. Park et at., Biology (Basel) 10(7), 610 (2021).

In a syngeneic murine colon carcinoma BALB/c murine model using MSS cell line CT-26, we observed significantly improved survival of mice treated with elraglusib and anti-PD-L1 therapy. We also demonstrated increased survival of mice treated with elraglusib alone as compared to the control group. We also observed statistically significant improved survival in the anti-PD-1 and anti-PD-L1 alone groups as compared to the control. Responders had lower percentages of splenic CD4+ T cells and splenic CD8+ T cells and had increased percentages of CD69+ activated T cells and Foxp3+ Tregs. The increased splenic percentages of both activated and end-stage T cells in the responder groups could be indicative of an anti-tumor immune response that was mounted earlier in the treatment course. Future studies could analyze the changes in these immune cell populations during the course of therapy in greater detail, especially considering we could have missed important changes in immune cell subtypes due to limited timepoints. Compared to non-responders, responders also had more CD3+ and CD4+ tumor-infiltrating lymphocytes. Further studies could evaluate the contribution of CD4+ versus CD8+ tumor-infiltrating T cells to the observed response to elraglusib and anti-PD-L1 therapy, especially considering the recent interest in the contribution of CD4+ helper T cells to anti-tumor immunity. We did not observe many significant differences in splenic NK cell subpopulations in either the tumor or the spleen, although perhaps the timepoint we chose to analyze was not representative of NK cell subpopulation changes that may have occurred earlier or later in the course of treatment. One limitation of this model is that it is a heterotopic flank tumor model as opposed to an orthotopic colon tumor model which may be more representative of the CRC TME. Follow-up experiments could examine the contribution of CD4+ T cells, CD8+ T cells, and NK cells to response to therapy in the murine MSS CRC model by blocking the function of each cell population in individual experiments.

We observed that murine responders had lower serum concentrations of BAFF, CCL7, CCL12, VEGF, VEGFR2, and CCL21. BAFF is a cytokine that belongs to the TNF ligand superfamily, that may promote tumorigenesis indirectly by induction of inflammation in the TME and directly by induction of EMT. Meanwhile, CCL7 has been shown to enhance both cancer progression and metastasis via EMT, including in CRC cells. Similarly, others have demonstrated that CXCR4 plays a critical role in the promotion of the progression of inflammatory CRC. It is commonly known that expression of VEGF-1 in CRC is associated with disease localization, stage, and long-term survival. We had previously observed suppression of VEGF in a panel of CRC cell lines post-elraglusib treatment and saw a similar suppression of VEGF in the murine responders. Moreover, we noted a decrease in VEGFR2 in murine responders, a protein that is highly expressed in CRC and promotes angiogenesis. Finally, CCL21 has been shown to play a role in colon cancer metastasis. Since many of the downregulated analytes in responders play a role in EMT, future studies of elraglusib could include metastatic CRC models.

We observed that responders had higher serum concentrations of CCL4, TWEAK, GM-CSF, CCL22, and IL-12p70 as compared to non-responders. Others have demonstrated that CCL4 is an important chemokine in the TME in determining response to ICB and that a lack of CCL4 can lead to the absence of CD103+ dendritic cells (DCs). DCs are an important cell population influencing the response to ICB, and although we did not monitor their levels in this study, it is conceivable that they played a role in influencing response to therapy. For this reason, further studies could monitor DC populations during the course of therapy. TWEAK is commonly expressed by peripheral blood monocytes and upregulates its expression after exposure to IFN-γ. TWEAK has also been shown to promote the nuclear translocation of both classical and alternative NF-κB pathway subunits. GM-CSF is a well-known immunomodulatory factor that has immunostimulatory functions but it is also predictive of poor prognosis in CRC. Finally, we observed increased levels of IL-12p70 in murine responders. IL-12 is a potent, pro-inflammatory cytokine that has been shown to increase activation and cytotoxicity of both T and NK cells as well as inhibit immunosuppressive cells, such as TAMs and myeloid-derived suppressor cells (MDSCs). We demonstrated that GSK-3 inhibitors such as elraglusib represent a possible combination strategy to increase the efficacy of ICB in patients with MSS CRC. The elraglusib-mediated increase in tumor surface cell-expressed PD-L1 presumably makes this an ideal small molecule to combine with anti-PD-L1 therapies. As this study was concerned solely with CRC, future studies could evaluate the combination of GSK-3 inhibitors with ICB in other malignancies of interest such as pancreatic cancer.

Cytokine analysis of plasma samples from patients with refractory solid tumors of multiple tissue origins enrolled in a Phase 1 clinical trial investigating elraglusib (NCT03678883) revealed that elevated baseline plasma levels of proteins such as IL-1β and reduced levels of proteins such as VEGF correlated with improved PFS and OS. PFS was also found to be positively correlated with elevated plasma levels of immunostimulatory analytes such as Granzyme B, IFN-γ, and IL-2 at 24 hours post-treatment with elraglusib. Several of these secreted proteins correlated with results from the in vivo study where expression of proteins such as IL-1β, CCL22, CCL4, and TWEAK was positively correlated with improved response to therapy while expression of proteins such as BAFF and VEGF negatively correlated with response to therapy. These results introduce novel circulating biomarkers for correlations with response to therapy which could provide significant clinical utility.

DSP analysis of paired FFPE tumor biopsies from patients with CRC or pancreatic cancer before and after treatment revealed that CD39 expression in PanCK+ segments was negatively correlated with duration of treatment while CD163 expression in CD45+ segments was positively correlated with duration of treatment and potential therapeutic benefit. It is known that CD39 can inhibit costimulatory signaling, increase immunosuppression during T cell priming, and its expression is associated with TAMs, Tregs, and inhibited cytotoxic immune cell function (46). CD39 has been shown to suppress pyroptosis, impair immunogenic cell death, and CD39 expression on endothelial cells regulates the migration of immune cells and promotes angiogenesis. Moreover, CD163 is a marker of cells from the monocyte/macrophage lineage therefore future studies could evaluate the impact of monocyte/macrophages on response to elraglusib. We also noted that immune cell segments showed differential protein expression based on the proximity to the tumor where tumor-infiltrating immune cells had decreased expression of immune checkpoints (PD-L1, Tim-3, PD-1) and Treg markers (CD25, CD127) as compared to tumor-adjacent immune cells regardless of timepoint. While the downregulation of immune checkpoint proteins PD-1, TIGIT, and LAG-3 by elraglusib has been previously described in melanoma models (47), our findings regarding VISTA and PD-L2 have not yet been reported. These novel observations regarding emerging immune checkpoint inhibitors should be included in future correlative studies regarding GSK-3 inhibition.

When we analyzed differential protein expression between Long Tx patients and Short Tx patients, we found that Long Tx patients had lower post-treatment expression of mature B cell/DC marker CD35, antigen NY-ESO-1, antigen Her2, antigen MART1, cytotoxic T cell marker CD8, Treg marker Foxp3, antigen PTEN, DC/myeloid marker CD11c, memory T cell marker CD45RO, checkpoint PD-L1, and PR in PanCK+ segments as compared to Short Tx patients which introduces several novel potential biomarkers of response to GSK-3 therapy which should be validated in further studies. Moreover, when we compared post-treatment protein expression in tumor-infiltrating CD45+ immune cell segments in Long Tx patients and Short Tx patients and found that Long Tx patients had decreased expression of antigens NY-ESO-1, PTEN, and PR as compared to Short Tx patients. Interestingly, these three antigens (NY-ESO-1, PTEN, and PR) had decreased expression in Long Tx patients post-treatment regardless of tumor or immune cell region.

In conclusion, this work demonstrates that small-molecule inhibition of GSK-3 using elraglusib may be a potential means to increase the efficacy of ICB and improve response in patients with MSS CRC, and possibly other tumor types. These findings support further studies and clinical development of elraglusib in combination with ICB, anti-PD-L1 therapy in particular. Moreover, this study, to our knowledge, represents the first digital spatial analysis of tumor biopsies from patients treated with elraglusib and very few oncology drugs have been evaluated using GeoMx technology to date. The novel circulating biomarkers of response to GSK-3 inhibition identified using the cytokine profiling data could provide significant clinical utility and the spatial proteomics data gives us novel insights into the immunomodulatory mechanisms of GSK-3 inhibition.

### Methods

### Cell Culture Maintenance

Human CRC cells SW480 (RRID: CVCL_0546), HCT-116 (RRID: CVCL_0291), HT-29 (RRID: CVCL_0320), and KM12C (RRID: CVCL_9547) were used in this study. SW480 cells were cultured in Dulbecco's Modified Eagle Medium (DMEM) supplemented with 10% FBS and 1% Penicillin-Streptomycin HCT-116 and HT-29 were cultured in McCoy's 5A (modified) Medium supplemented with 10% FBS and 1% Penicillin-Streptomycin. KM12C cells were cultured in Eagle's Minimal Essential Medium supplemented with 10% FBS and 1% Penicillin-Streptomycin. Human immune cells NK-92 (RRID: CVCL_2142), TALL-104 (RRID: CVCL_2771), and patient-derived CD8+ T cells were also used in this study. NK-92 cells were cultured in Alpha Minimum Essential medium supplemented with 2 mM L-glutamine, 1.5 g/L sodium bicarbonate, 0.2 mM inositol, 0.1 mM 2-mercaptoethanol, 0.02 mM folic acid, 12.5% horse serum, and 12.5% FBS. TALL-104 cells (CD2+; CD3+; CD7+; CD8+; CD56+; CD4−; CD16−) and patient-derived T cells (CD3+; CD8+) were cultured in RPMI-1640 containing 20% FBS, 100 U/ml penicillin, and 100 μg/ml streptomycin. Recombinant human IL-2 (Miltenyi cat #130-097744) with a final concentration of 100 units/mL was added to all immune cell culture media. All cell lines were incubated at 37° C. in a humidified atmosphere containing 5% CO₂. Cell lines were authenticated and tested to ensure the cultures were free of mycoplasma infection.

### Measurement of Cell Viability

Cells were seeded at a density of 3×10³ cells per well in a 96-well plate (Greiner Bio-One, Monroe, NC, USA). Cell viability was assessed using the CellTiter Glo assay (Promega, Madison, WI, USA). Cells were mixed with 25 μL of CellTiter-Glo reagents in 100 μL of culture volume, and bioluminescence imaging was measured using the Xenogen IVIS imager (Caliper Life Sciences, Waltham, MA). The percent of cell viability was determined by normalizing the luminescence signal to control wells. Dose-response curves were generated and the half maximal inhibitory concentration (IC-50) was calculated using Graph-Pad Prism (RRID: SCR_002798) version 9.2.0. For IC50 generation, concentrations were log-transformed and data were then normalized to control and a log (inhibitor) versus response (three parameters) test was used.

### Pyroptosis Assay

Recombinant Human TNF-α (Cat #300-01A, PeproTech, Rocky Hill, NJ, USA) and Recombinant Human IFN-γ (Cat #300-02, Peprotech, Rocky Hill, NJ, USA) were purchased for use in western blot analysis while rhTRAIL was generated in-house. Kim et al., J Biol Chem., 279(38):40044-52 (2004).

| Reagent or Resource | Source | Identifier | Dilution |
|---|---|---|---|
| Vinculin (E1E9V) XP ® Rabbit mAb | Cell Signaling | Cat# 13901 | 1:1000 |
| Anti-GSDMB antibody | Sigma-Aldrich | Cat# HPA052407 | 1:1000 |

### Isolation of Donor-Derived CD8+ T Cells

An Easy Step Human CD8+ T Cell Isolation Kit was used to isolate CD8+ T cells from a donor PBMC sample via negative selection (Cat #, 17913, Stem Cell Technologies, Vancouver, Canada).

### Collection of Cell Culture Supernatants Used in Cytokine Measurements

Cells were plated at 3.5×10⁴ cells in a 48-well plate (Thermo Fisher Scientific, Waltham, MA, USA) in complete medium and incubated at 37° C. with 5% CO₂. At 24 hours after plating, almost all the tumor cells were adherent to the bottom of the flask and the complete medium was replaced with the drug-containing medium. Subsequently, the culture supernatants were collected after 48 hours of incubation and were frozen at −80° C. until the measurement of cytokines was performed. On the day of analysis, samples were thawed and centrifuged to remove cellular debris.

### Human Cytokine Profiling

Human cell line culture supernatants were analyzed using an R&D systems Human Premixed Multi-Analyte Kit (R&D Systems, Inc., Minneapolis, MN, USA) and a Luminex 200 (RRID: SCR_018025) Instrument (LX200-XPON-RUO, Luminex Corporation, Austin, TX, USA) according to the manufacturer's instructions. Sample levels of TNF-α, 4-1BB/TNFRSF9/CD137, IL-8/CXCL8, Ferritin, IFN-β, IL-10, CCL2/JE/MCP-1, VEGF, CXCL13/BLC/BCA-1, IFN-γ, CCL20/MIP-3 α, CCL3/MIP-1 α, CCL22/MDC, CCL4/MIP-1 β, Fas Ligand/TNFSF6, IL-17/IL-17A, IL-2, BAFF/BLyS/TNFSF13B, GM-CSF, CXCL5/ENA-78, TRANCE/TNFSF11/RANK L, CXCL9/MIG, G-CSF, IFN-γ R1/CD119, VEGFR3/Flt-4, C-Reactive Protein/CRP, CXCL11/I-TAC, IL-21, CXCL14/BRAK, IL-6, Fas/TNFRSF6/CD95, TRAIL R3/TNFRSF10C, IL-4, CCL5/RANTES, PD-L1/B7-H1, CCL7/MCP-3/MARC, Chitinase 3-like 1, CXCL10/IP-10/CRG-2, IL-1 β/IL-1F2, IL-7, Prolactin, CCL8/MCP-2, TRAIL R2/TNFRSF10B, M-CSF, IL-15, Granzyme B, IFN-α, TREM-1, IL-12/IL-23 p40, TRAIL/TNFSF10, CCL11/Eotaxin, and IL-18/IL-1F4. Quantitative analysis with 6 standards and a minimum of 50 counts per bead region was used with the Luminex to generate analyte values reported as picograms/milliliter (pg/mL). Sample concentrations less than the lower limit of detection for each particular analyte were recoded as the lower limit value divided by ten. Sample concentrations above the upper limit of detection for a particular analyte were recoded as the upper limit of detection.

### Murine Cytokine Profiling

Whole blood from mice was collected, allowed to clot, and serum was isolated using a serum separator tube (SST) according to manufacturer instructions. Murine serum samples were analyzed using an R&D systems Murine Premixed Multi-Analyte Kit (R&D Systems, Inc., Minneapolis, MN, USA) and a Luminex 200 (RRID: SCR_018025) Instrument (LX200-XPON-RUO, Luminex Corporation, Austin, TX, USA) according to the manufacturer's instructions. Sample levels of GM-CSF, IL-7, IL-12 p70, CCL2/JE/MCP-1, IL-1 β/IL-1F2, VEGF, IL-2, IL-4, VEGFR2/KDR/Flk-1, IL-6, IL-10, IL-13, IFN-γ, IL-3, IL-16, CXCL10/IP-10/CRG-2, CCL5/RANTES, CCL7/MCP-3/MARC, CCL12/MCP-5, Prolactin, M-CSF, CCL3/MIP-1 α, IL-1 α/IL-1F1, CCL20/MIP-3 α, CCL4/MIP-1 β, TWEAK/TNFSF12, CXCL12/SDF-1 α, BAFF/BLyS/TNFSF13B, Granzyme B, CCL21/6Ckine, CCL11/Eotaxin, and CCL22/MDC. Sample values are reported in picograms per milliliter (pg/mL). Quantitative analysis with 6 standards and a minimum of 50 counts per bead region was used with the Luminex to generate analyte values reported as picograms/milliliter (pg/mL). Sample concentrations less than the lower limit of detection for each particular analyte were recoded as the lower limit value divided by ten. Sample concentrations above the upper limit of detection for a particular analyte were recoded as the upper limit of detection. Data analysis and visualization were generated using R (RRID: SCR_001905) software (R Development Core Team, 2020).

### GFP+ Cell Line Generation

50,000 HT-29 or HCT 116 cells were seeded in a 12-well tissue culture plate and allowed to adhere overnight. They were then transduced with lentivirus containing the plasmid pLenti_CMV_GFP_Hygro [pLenti CMV GFP Hygro (656-4) was a gift from Eric Campeau & Paul Kaufman (Addgene viral prep #17446-LV; RRID: Addgene_17446)] at a multiplicity of infection of 10 with 8 μg/mL polybrene (hexadimethrine bromide [Cat #107689, Sigma Aldrich, St. Louis, MO, USA) for 24 hours before washing with PBS and replacing with fresh medium (49). The cells were then sorted for GFP-positivity using a BD FACSAria™ III Cell Sorter (RRID: SCR_016695).

### Multicolor Immune Cell Co-Culture Experiments

10,000 HCT-116, SW480, or HT-29 cells were plated per well in a clear-bottom, black-walled 48-well tissue culture plate and were allowed to adhere overnight. Cells were subsequently treated with DMSO, 5 μM or 10 μM elraglusib, and/or 10,000 TALL-104 or NK-92 cells (for an effector-to-tumor ratio of 1:1) for 24 hours. CRC cells were labeled using CellTracker™ Green CMFDA (5-chloromethylfluorescein diacetate), immune cells (NK-92, TALL-104) were labeled using CellTracker™ Blue CMAC Dye (7-amino-4-chloromethylcoumarin), and ethidium homodimer-1 (EthD-1) was used as a marker of cell death (Invitrogen, Waltham, MA). 10× images were captured using a Nikon Ti-U Inverted Fluorescence Microscope and NIS-Elements F Package imaging software 3.22.00 Build 710 (Nikon Instruments Inc, USA). The number of red/green color cells in random fields was determined using thresholding and particle analysis in the Fiji modification (RRID: SCR_002285) of ImageJ and expressed as a dead/live cell ratio. Normalization was carried out by subtracting the percentage of cell death due to drug or vehicle control (DMSO) only from the percentage of dead cells observed in the co-culture of tumor and immune cells treated with the drug. At least 100 cells were evaluated per sample, with 3 independent replicates. Statistical analysis was done using GraphPad Prism 9 (RRID: SCR_002798).

### Single-Color Immune Cell Co-Culture Experiments

5000 HT-29 GFP+ or HCT 116 GFP+ cells were plated per well in a clear-bottom, black-walled 96-well tissue culture plate and were allowed to adhere overnight. Cells were subsequently treated with DMSO, 5 μM elraglusib, and/or 5000 TALL-104 or NK-92 cells (for an effector-to-tumor ratio of 1:1) for 48 hours. Nine images were taken per well at 10× magnification using a Molecular Devices ImageXpress® Confocal HT.ai High-Content Imaging System and quantified for the number of GFP+ objects using the MetaXpress (RRID: SCR_016654) software (Molecular Devices, San Jose, CA, USA). 40× Images were also taken at 24 hours for representative images of cellular morphology changes. Statistical analysis was done using GraphPad Prism 9 (RRID: SCR_002798).

### Generation of Single-Cell Suspensions

Spleens were strained, filtered, and washed while tumors were collected, washed, and digested before lymphocytes were collected using a Percoll gradient (Cat #P1644-100ML, Sigma Aldrich, St. Louis, MO).

### Flow Cytometry

Flow cytometry viability staining was conducted by suspending murine spleen and tumor single cell suspensions in Zombie Violet fixable viability kit (Cat #423114, BioLegend, San Diego, CA, USA) according to manufacturer instructions for 30 minutes at room temperature. Staining for membrane surface proteins was conducted using conjugated primary antibodies for 1 hour on ice, according to manufacturer instructions. Cells were fixed and permeabilized using the eBioscience™ Foxp3/Transcription Factor Staining Buffer Set according to manufacturer instructions (Cat #00-5523-00, Invitrogen, Waltham, MA). Cells were resuspended in Flow Cytometry Staining Buffer (R&D Systems, Minneapolis, MN, USA) and were analyzed using a BD Biosciences LSR II (RRID: SCR_002159) and FlowJo (RRID: SCR_008520) version 10.1 (FlowJo, Ashland, OR, USA).

| | | Catalog | |
|---|---|---|---|
| Antibody name | Manufactuer | number | Concentration |
| Zombie Violet ™ Fixable Viability Kit | BioLegend | 423114 | 1:1000 |
| CD45 Monoclonal Antibody (30-F11), eVolve 605 | eBioscience ™ | 83-0451-42 | 5 μL/test |
| PE Rat Anti-Mouse CD3 Molecular Complex, Clone | BD | 555275 | 0.125 μg/test |
| 17A2 (RUO) | Biosciences | | |
| CD335 (NKp46) Monoclonal Antibody (29A1.4), APC | eBioscience ™ | 17-3351-82 | 0.125 μg/test |
| APC/Cy7 anti-mouse/human CD11b, clone: M1/70 | BioLegend | 101226 | 0.125 μg/test |
| Cd27 Monoclonal Antibody (LG.7F9), FITC | eBioscience ™ | 11-0271-82 | 0.5 μg/test |
| Klrg1 Monoclonal Antibody (2F1), PE-Cyanine7 | eBioscience ™ | 25-5893-82 | 0.25 μg/test |
| Anti-mouse CD45, eBioscience, eVolve 605, clone: | Invitrogen | 83-0451-42 | μg/test |
| 30-F11 | | | |
| APC-Cy ™7 Rat Anti-Mouse CD3 Molecular Complex, | BD | 560590 | 0.125 μg/test |
| clone 17A2 | Biosciences | | |
| CD4 Monoclonal Antibody (RM4-5), PE-Cyanine7 | Invitrogen | 25-0042-82 | 0.25 μg/test |
| PE Rat Anti-Mouse CD8a, Clone 53-6.7 (RUO) | BD | 553032 | 0.125 μg/test |
| | Biosciences | | |
| CD69 Monoclonal Antibody (H1.2F3), FITC | eBioscience ™ | 11-0691-81 | 0.5 μg/test |
| FOXP3 Monoclonal Antibody (FJK-16s), APC | Manufacturer | 17-5773-82 | 1 μg/test |

### Natural Killer Cell Immunophenotyping

The NK cell flow cytometry panel included the following directly-conjugated primary antibodies: Anti-mouse CD45, eBioscience eVolve 605 clone: 30-F11 (Ref #83-0451-42, Invitrogen), PE anti-mouse CD3 molecular complex (17A2) (mat. #: 555275, BD biosciences), Anti-mouse NKp46 APC (Ref #17-3351-82), APC/Cy7 anti-mouse/human CD11b clone: M1/70 (cat #101226, BioLegend), anti-Cd27 Monoclonal Antibody (LG.7F9) FITC (eBioscience™, Thermo Scientific, cat #11-0271-82), and (Klrg1 Monoclonal Antibody (2F1) PE-Cyanine7 (eBioscience, Thermo Scientific, cat #25-5893-82). Gating strategies are as follows:
NK cell: live/CD45/CD3−/NK1.1+
Mature NK cell: live/CD45/CD3−/NK1.1+/KRLG1+
Activated NK cell: live/CD45/CD3−/NK1.1+/CD11b+
NK cell subset 1: live/CD45/CD3−/NK1.1+/CD11b-CD27−
NK cell subset 2: live/CD45/CD3−/NK1.1+/CD11b−CD27+
NK cell subset 3: live/CD45/CD3−/NK1.1+/CD11b+CD27+
NK cell subset 4: live/CD45/CD3−/NK1.1+/CD11b+CD27−

### T Cell Immunophenotyping

The T cell flow cytometry panel included the following directly-conjugated primary antibodies: Anti-mouse CD45 superbright 600 clone: 30-511 (ref #63-0451-82, eBioscience), anti-CD3 APC-Cy7 clone 17A2(BD Biosciences, cat #560590), eBioscience anti-mouse CD4 PE-Cy7 clone: RM4-5 (Ref #25-0042-82, Invitrogen), PE anti-mouse CD8a (Ly-2)(53-6.7) (cat #553032, BD), Anti-mouse CD69 FITC clone: H1.2F3 (Ref #11-0691-81, eBioscience), and Foxp3 (FJK-16s) APC (eBioscience). Gating strategies are as follows:
CD4+ T cell: live/CD45+/CD3+/CD4+/Foxp3−
CD8+ T cell: live/CD45+/CD3+/CD8+
Treg: live/CD45+/CD3+/CD4+/Foxp3+
Activated CD8+ T cell: live/CD45+/CD3+/CD8+/CD69+

### Western Blot Analysis

Cells were plated in a 6-well plate and incubated overnight before the spent media was replaced with drugged media. Drug treatment lasted for indicated durations. Protein was extracted using radioimmunoprecipitation (RIPA) assay buffer (Cat #R0278, Sigma-Aldrich, St. Louis, MO) containing cOmplete™, Mini, EDTA-free Protease Inhibitor Cocktail (Cat #4693159001, Roche, Basel, Switzerland) from sub-confluent cells. Denaturing sample buffer was added, samples were boiled at 95 degrees for 10 minutes, and an equal amount of protein lysate was electrophoresed through NuPAGE™ 4 to 12%, Bis-Tris, 1.5 mm, Mini Protein Gels (Invitrogen, Waltham, MA) then transferred to PVDF membranes. The PVDF membrane was blocked with 5% non-fat milk (Sigma-Aldrich, St. Louis, MO) in 1×TTBS. Primary antibodies were incubated with the transferred PVDF membrane in blocking buffer at 4° C. overnight. Secondary antibodies used included Goat anti-Rabbit IgG (H+L) Secondary Antibody, HRP (Cat #31460, Invitrogen, Waltham, MA), and Goat anti-Mouse IgG (H+L) Secondary Antibody, HRP (Cat #31430, Invitrogen, Waltham, MA). Signal was detected using Pierce™ ECL Western Blotting Substrate (Cat #32106, Thermo Scientific, Waltham, MA, USA) and a Syngene Imaging System (RRID: SCR_015770).

| Reagent or Resource | Source | Identifier | Dilution |
|---|---|---|---|
| PARP Antibody | Cell Signaling | Cat# 9542S | 1:1000 |
| Mcl-1 (D2W9E) Rabbit mAb | Cell Signaling | Cat# 94296S | 1:1000 |
| NF-KB p65 (L8F6) Mouse mAb | Cell Signaling | Cat# 6956 | 1:1000 |
| PD-L1 (E1L3N ®) XP ® Rabbit mAb | Cell Signaling | Cat# 13684 | 1:1000 |
| Bcl-2 (D55G8) Rabbit mAb | Cell Signaling | Cat# 4223S | 1:1000 |
| Survivin (71G4B7) Rabbit mAb | Cell Signaling | Cat# 2808S | 1:1000 |
| Mouse Anti-Ran | BD Biosciences | Cat# 610341 | 1:5000 |
| NIK Antibody | Cell Signaling | Cat# 4994 | 1:1000 |

### In Vivo Studies

The experimental in vivo protocol (Protocol #19-01-003) was approved by the Institutional Animal Care and Use Committee of Brown University (Providence, RI, USA). Six to 7 weeks-old female BALB/c mice (RRID: IMSR_JAX:000651) were purchased from Taconic. 50,000 cells were suspended in 50 μL ice-cold PBS and 50 μL Matrigel (Catalog # 354234, Corning, New York, USA), and 100 uL was injected subcutaneously into the rear flanks. Once tumor volume reached at least 100 mm³, mice were randomly assigned to one of seven groups (3 mice/group): Control (isotype), elraglusib, elraglusib+Isotype, anti-PD-1, anti-PD-L1, elraglusib+anti-PD-1, and elraglusib+anti-PD-L1. All treatments were delivered by IP injection on the following dosing schedule: Isotype (70 mg/kg, twice a week), elraglusib (70 mg/kg, twice a week), anti-PD-1 (10 mg/kg, twice a week), anti-PD-L1 (10 mg/kg, twice a week). The treatment continued until mice developed signs of discomfort from excessive tumor growth. Mice were weighed once a week to monitor signs of drug toxicity. The length (L) and width (W) of the masses were measured three times per week with a digital caliper, and the tumor volume was calculated by applying the formula: 0.5LW2. Collection of whole blood and serum was performed by cardiac puncture and sent to Antech GLP for blood cell count and chemistry tests, or in-house cytokine profiling. Tumors and organs were dissected and harvested for analysis by IHC and flow cytometry.

### Immunohistochemistry

Excised tissues are fixed with 10% neutral buffered formalin and paraffin-embedded. 5-micrometer tissue sections are cut with a microtome and mounted on glass microscope slides for staining. Hematoxylin and eosin staining was completed for all tumor specimens. Paraffin embedding and sectioning of slides were performed by the Brown University Molecular Pathology Core Facility. Slides were dewaxed in xylene and subsequently hydrated in ethanol at decreasing concentrations. Antigen retrieval was carried out by boiling the slides in 2.1 g citric acid (pH 6) for 10 minutes. Endogenous peroxidases were quenched by incubating the slides in 3% hydrogen peroxide for 5 minutes. After nuclear membrane permeabilization with Tris-buffered saline plus 0.1% Tween 20, slides were blocked with horse serum (Cat #MP-7401-15, Vector Laboratories, Burlingame, CA, USA), and incubated with primary antibodies overnight (Supplementary Table S1) in a humidified chamber at 4C. After washing with PBS, a secondary antibody (Cat #MP-7401-15 or MP-7402, Vector Laboratories, Burlingame, CA, USA) was added for 30 minutes, followed by diaminobenzidine application (Cat #NC9276270, Thermo Fisher Scientific, Waltham, MA, USA) according to the manufacturer's protocol. Samples were counterstained with hematoxylin, rinsed with distilled water, dehydrated in an increasing gradient of ethanol, cleared with xylene, and mounted with Cytoseal mounting medium (Thermo Fisher Scientific, catalog no. 8312-4). Images were recorded on a Zeiss Axioskop microscope (RRID: SCR_014587), using QCapture (RRID: SCR_014432). QuPath software (RRID: SCR_018257) was used to automatically count positive cells. For each IHC marker, five 20× images per group were analyzed, and results were represented as the absolute number of positive cells per 20× field.

| | | Catalog | Antibody |
|---|---|---|---|
| Antibody name | Manufacturer | number | dilution |
| CD4 (D7D2Z) Rabbit mAb | Cell Signaling | 25229S | 1:200 |
| CD8a (D4W2Z) XP ® Rabbit mAb (Mouse Specific) | Cell Signaling | 98941 | 1:800 |
| Anti-TRAIL antibody | Abcam | ab231265 | 20 μg/ml |
| NKp46 (CD335) Polyclonal Antibody | Invitrogen | PA5-79720 | 1 μg/mL |
| FoxP3 (D6O8R) Rabbit mAb | Cell Signaling | 12653 | 1:800 |
| Granzyme B (E5V2L) Rabbit mAb | Cell Signaling | 44153 | 1:200 |
| Ki-67 (D3B5) Rabbit mAb (Mouse Preferred; IHC | Cell Signaling | 12202 | 1:800 |
| Formulated) | | | |
| PD-1/CD279 Polyclonal antibody | Proteintech | 18106-1-AP | 1:1000 |
| PD-L1/CD274 Monoclonal antibody | Proteintech | 66248-1-Ig | 1:5000 |
| Cleaved Caspase-3 (Asp175) Antibody | Cell Signaling | 9661 | 1:400 |
| VEGF Monoclonal Antibody (JH121) | Invitrogen | MA5-13182 | 1:20 |
| TGF beta 2-Specific Polyclonal antibody | Proteintech | 19999-1-AP | 1:500 |

### Microarrays

A total of 0.5×10⁶ tumor cells (HCT-116, HT-29, KM12C) were plated in a 6-well plate and allowed to adhere overnight before 24-hour treatment as indicated. 1×10⁶ immune cells (NK92, TALL-104) were plated and treated with elraglusib as indicated for 24 hours. RNA was isolated from cell pellets in batches of 6 using an RNeasy Plus Mini Kit (Cat #74134, Qiagen, Hilden, Germany). Acceptable RNA concentration and quality were verified with Nanodrop and Bioanalyzer measurements. GeneChip™ Human Transcriptome Array 2.0 assays were conducted according to manufacturer instructions in two batches using randomized samples to limit batch effects (Cat #902162, Applied Biosystems, Waltham, MA, USA). Applied Biosystems Transcriptomic Analysis Console (TAC) software (RRID: SCR_016519) was used to calculate fold changes in gene expression relative to the untreated control cells. Values were considered statistically significant for p values <0.05.

### Single-Cell RNA Sequencing

Single cells were captured and 3′ single-cell gene expression libraries were conducted (Next GEM v3.1) using the 10× Genomics Chromium system by SingulOmics (SingulOmics, New York, NY, USA). Gene expression libraries were sequenced with ~200 million PE150 reads per sample on Illumina (RRID: SCR_016387) NovaSeq (Illumina, Inc., San Diego, CA, USA). After sequencing clean reads were then analyzed with human reference genome GRCh38 using Cell Ranger v6.1.2 ([RRID: SCR_017344],10× Genomics, Pleasanton, CA, USA). Data were analyzed and visualized using Loupe Browser ([RRID: SCR_018555], 10× Genomics, Pleasanton, CA, USA).

### Digital Spatial Profiling

An Agilent technologies hybridization oven was used for baking tissue onto slides (Agilent, Santa Clara, CA, USA). A NanoString GeoMx® Digital Spatial Profiler (DSP) instrument (NanoString, Seattle, WA, USA) was used to scan slides, identify regions of interest (ROIs), and collect photocleavable barcodes according to manufacturer instructions. A custom panel was designed to include the following proteins: Ms IgG1, Ms IgG2a, Rb IgG, GAPDH, Histone H3, S6, Beta-2-microglobulin, CD31, CD45, Ki-67, ARG1, CD11b, CD11c, CD14, CD163, CD39, CD40, CD68, HLA-DR, GZMB, CD20, CD3, CD34, CD4, CD56, CD66b, CD8, Foxp3, Fibronectin, 4-1BB, B7-H3, CTLA4, GITR, IDO1, LAG3, OX40L, STING, Tim-3, VISTA, Bcl-2, ER-α, EpCAM, Her2, MART1, NY-ESO-1, PR, PTEN, PanCk, SMA, CD127, CD25, CD27, CD44, CD45RO, CD80, ICOS, PD-1, PD-L1, and PD-L2. An Eppendorf MasterCycler Gradient Thermal Cycler was used to generate the Illumina sequencing libraries from the photocleaved tags. (Eppendorf, Hamburg, Germany). An Agilent Fragment Analyzer (RRID: SCR_019417) was used for library size distribution analysis with a high-sensitivity NGS Fragment Kit (Cat #DNF-474-0500, Agilent, Santa Clara, CA, USA). qPCR for quantification was run using an Illumina-compatible KAPA Library Quantification Kits (ROX Low) (cat #KK4873) on an Applied Biosystems ViiA 7 Real-Time qPCR/PCR Thermal Cycler System (Applied Biosystems, San Francisco, CA, USA) and was analyzed using QuantStudio software (RRID: SCR_018712). Sequencing was performed using a NextSeq 500/550 High Output Kit v2.5 (75 Cycles) kit (cat #20024906) on an Illumina Sequencing NextSeq 550 System ([RRID: SCR_016381], Illumina, San Diego, CA, USA). The initial annotated dataset went through quality control (QC) to check if housekeeper genes and background (isotype) control molecules were themselves correlated with the predictors of interest. Every ROT was tested for raw sequencing reads (segments with <1000 raw reads were removed), % sequencing saturation (defined as [1-deduplicated reads/aligned reads]%, segments below ~50% were not analyzed), and nuclei count per segment (>100 nuclei per segment is generally recommended). Both immunoglobulins (IgGs) and housekeeper genes were highly correlated with one another. Signal to noise (SNR) ratio was calculated using background probes and all probes were detected above the background in at least one ROI. Finally, data were normalized based on background IgG expression and all normalization factors were well distributed. Data analysis and visualization were generated using R ([RRID: SCR_001905], R Development Core Team, 2020).

### Clinical Specimens

Archival tumor specimens and peripheral blood samples were collected from patients enrolled in the Phase I study of Elraglusib (9-ING-41), a small molecule selective glycogen synthase kinase-3 beta (GSK-3b) inhibitor, as monotherapy or combined with cytotoxic regimens in patients with relapsed or refractory hematologic malignancies or solid tumors (Clinicaltrials.gov NCT03678883) who received treatment at the Lifespan Cancer Institute (Providence, RI, USA). The study was conducted in accordance with the Declaration of Helsinki and the International Conference on Harmonization Good Clinical Practice guidelines. The study protocol was approved by the Institutional Review Board (IRB) of Rhode Island Hospital under protocol number 1324888-120. The patients also participated in a Lifespan Cancer Institute research protocol designed to investigate molecular and genetic features of tumors and mechanisms of resistance (Rhode Island Hospital IRB protocol number 449060-38). All patients provided written informed consent.

### Statistical Analysis

GraphPad Prism (RRID: SCR_002798) version 9.5.0 was used for statistical analyses and graphical representation (GraphPad, San Diego, CA, USA). Data are presented as means±standard deviation (SD) or standard error of the mean (SEM). The relations between groups were compared using two-tailed, paired student's T tests or one-way ANOVA tests. Survival was analyzed with the Kaplan-Meier method and was compared with the log-rank test. For multiple testing, Tukey's or Benjamini-Hochberg's methods were employed. Statistical significance is reported as follows: P≤0.05: *, P≤0.01: **, and P≤0.001: ***.

The complete disclosure of all patents, patent applications, and publications, and electronically available materials cited herein are incorporated by reference. The foregoing detailed description and examples have been given for clarity of understanding only. No unnecessary limitations are to be understood therefrom. In particular, while various theories are presented describing possible mechanisms through with the compounds are effective, the compounds are effective regardless of the particular mechanism employed and the inventors are therefore not bound by theories described herein. The invention is not limited to the exact details shown and described, for variations obvious to one skilled in the art will be included within the invention defined by the claims.

What is claimed is:

1. A method of treating cancer in a subject in need thereof, comprising administering to the subject a therapeutically effective amount of a maleimide derivative while also treating the subject with an immune checkpoint blockade, wherein the maleimide derivative is according to formula I: 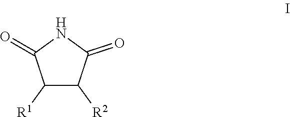
wherein R¹ and R² are alkyl or aryl groups, or a pharmaceutically acceptable salt thereof.

2. The method of claim 1, wherein the cancer is a low PD-L1 expressing cancer.

3. The method of claim 1, wherein the cancer is colorectal cancer.

4. The method of claim 1, wherein the cancer is microsatellite stable.

5. The method of claim 1, wherein the subject is human.

6. The method of claim 1, wherein the maleimide derivative is a GSK-3 inhibitor.

7. The method of claim 1, wherein the maleimide derivative is administered together with a pharmaceutically acceptable carrier.

8. The method of claim 1, wherein are R¹ and R² are C₆-C₁₂ alkyl or aryl groups.

9. The method of claim 1, wherein R¹ and R² are C₆-C₁₂ aryl groups.

10. The method of claim 1, wherein R¹ and R² are C₆-C₁₂ heteroaryl groups.

11. The method of claim 1, wherein the maleimide derivative is a compound selected from the group consisting of NSC767225, NSC767202, NSC767238, NSC59984, NSC767276, NSC767334, NSC767335, and 9-ING-41.

12. The method of claim 1, wherein the immune checkpoint blockade comprises PD-L1 inhibition.

13. The method of claim 1, wherein the maleimide derivative and the immune checkpoint blockade are provided simultaneously.
